(19) United States
(12) Patent Application Publication
(10) Pub. No.: US 2014/0142200 A1
(43) Pub. Date: May 22, 2014

(54) Keratoprosthesis

(71) Applicant: Eyegenix LLC, Honolulu, HI (US)

(72) Inventors: Xiaodong Duan, Honolulu, HI (US); Priscilla Ann Carbajal, Waimanalo, HI (US); Anthony Lee, San Francisco, CA (US)

(73) Assignee: Eyegenix LLC

(21) Appl. No.: 13/678,567

(22) Filed: Nov. 16, 2012

(51) Int. Cl. A61L 27/24; A61L 15/32; A61K 47/42

(52) U.S. Cl. CPC A61L 27/24; A61K 47/42; A61L 15/325
USPC 514/773

(57) ABSTRACT

The invention comprises a method of making molded, double-crosslinked (i.e., two stages of crosslinking), transparent, collagen materials using a novel combination of diafiltration, lyophilization, and homogenization. The collagen material can be used not only as an ophthalmic device, but also as a tissue scaffold, drug delivery device, wound dressing, or other collagen hydrogel based device.

### BACKGROUND OF THE INVENTION

1. Field of the Invention

The present invention is in the field of crosslinked collagen used to make prosthetic devices, tissue substitutes and scaffolding, drug delivery devices, and ophthalmic devices, and particularly relates to keratoprostheses made using multi-stage crosslinked collagen.

2. Related Art

Keratoprotheses have been studied for years to treat corneal blindness, the typical therapy for which comprises suturing a single-stage crosslinked, bioengineered polymeric, “core-and-porous skirt” design, keratoprothesis (“kPro”) to replace (in whole or in part) a diseased or injured cornea. However, inflammatory- and detachment-related complications have slowed the adoption of kPros and reflect the need for improving the biocompatibility of the materials used in making kPros. In the native cornea, the stromal layer accounts for 90% of the corneal thickness, with the extracellular matrix (“ECM”) comprising up to 85% of the stroma. The major components of the stromal ECM are collagen (Type I) and glycosaminoglycans.

Collagen has long been studied as a bioengineering scaffold, including its use to biointegrate into corneal tissue (i.e., into Bowman's layer, remaining stroma, and Descemet's membrane) after the replacement or supplementation of a diseased or damaged cornea. A “full thickness” kPro is used to replace the stroma in a cornea. A “supplementation” kPro refers to an overlay, inlay, or underlay (defined below), which interfaces with stroma not removed from a patient's cornea.

Small molecule, chemical crosslinkers like EDC (1-ethyl-3′-(3-dimethylaminopropyl) carbodiimide, aka EDAC or EDC), along with NHS (N-hydroxy succinimide), have been widely used to crosslink proteins (e.g., collagen) for biomedical applications. “NH₂” is used herein as an abbreviation for “collagen primary amine group” and represents the molar value of primary amine group of collagen used in the reaction mixture. The “reaction mixture” comprises crosslinker and collagen solution.

A typical “single-stage” method of using collagen to make a kPro utilizes crosslinkers to crosslink recombinant human collagen type I or III (initial concentration of up to 12% w/w (weight per weight is herein abbreviated “w/w”; weight per volume is herein abbreviated “w/v”). The crosslinker is mixed with collagen to form a reaction mixture that is injected into cornea-shaped molds; the crosslinking reaction takes place in a single stage (a “stage” is the reaction period following a given mixing of collagen and crosslinker); and cornea-shaped hydrogels are released from the molds. The crosslinker molar ratio is selected to optimize the properties of a collagen hydrogel produced in a single-stage crosslinking reaction. A commonly used reaction mixture molar ratio is 5/5/1 of EDC/NHS/NH₂ when a lower collagen concentration (0.5-3%, w/w) is used. However, that reaction mixture typically gels (crosslinks) very quickly. When a higher collagen concentration (12%, w/w) is used, the fast reaction rate makes it impossible to use a reaction mixture molar ratio beyond 0.5/0.5/1. Adding more crosslinker makes the reaction mixture become too viscous to fill the molds; the reaction mixture solidifies inside the dispensing apparatus within seconds. In contrast, kPros produced using a lower reaction mixture molar ratio, e.g., 0.5/0.5/1, exhibit poor suture retention, durability, and collagenase resistance.

When higher collagen concentrations (e.g., 13.7%, up to 23.5%, w/w) and slightly higher EDC/NHS/NH₂ molar ratios (e.g., 0.7/0.7/1, to 1/1/1) were explored in an effort to improve mechanical strength and robustness of kPros to meet the requirements of suture retention and durability for full-thickness corneal implantation, the fast reaction resulting from the higher crosslinker concentrations made it very difficult to achieve hydrogels with homogeneous optical and acceptable mechanical properties. The fast crosslinking reaction makes the reaction mixture solidify too fast for proper injection into the molds, and the resultant kPros have non-uniform optical properties. All known kPros produced using single-stage crosslinking, EDC/NHS/NH₂ molar ratios higher than 0.5/0.5/1, and higher collagen concentrations have poor homogeneity, cloudiness, and poor mechanical properties.

Efforts have been made to use two stages of crosslinking to improve mechanical properties of collagen-based materials while preserving the optical performance required for ophthalmic devices. U.S. Pat. No. 4,931,546 (to Tardy, et al.) (“Tardy”) discloses a process for cross-linking collagen twice. In Example 3 of Tardy, Tardy mixed Type IV human placental collagen (“HPC”) (15%, w/w) with 0.0001M periodic acid (pH 7.5), placed the reaction mixture in a lenticular mold, allowed the reaction mixture to crosslink, removed the hydrogel lens from the mold, washed the lens, placed the washed lens in a solution of 0.01M sodium periodate (pH 7.5), washed the lens, and reported that the final product (after two stages of crosslinking) had the same mechanical and optical properties as a lens produced with one stage of crosslinking (the product of Tardy's Example 1). In Tardy's own words, “The lens or implant has the same characteristics as those described in Example 1.” (final sentence in Tardy's Example 3) Tardy's focus in his remaining Examples (4 to 17) is on fibrillar, collagen-based tissue substitutes and wound fillers. Applicant replicated Tardy's Examples 2 (single-stage crosslinking) and 3 (two stages of crosslinking), using both HPC Type IV, and Type I Collagen, without success: the final product using HPC Type IV collagen remained an amorphous glob of gel and could not be molded into a structure; the final product using Type I collagen completely dissolved in 0.01M sodium periodate (pH 7.5).

US Published Application 20110207671 (by Chang, et al.) (“Chang”) discloses a method for producing double-crosslinked collagen material using fibrillar collagen materials, without molding, at low collagen concentration (35 mg/ml), under neutral pH to basic pH, without possibility of use in ophthalmic devices (crosslinked, fibrillar collagen is an opaque or cloudy liquid/suspension), and without disclosure of mechanical and optical properties.

The technical problem to be solved is to produce a stronger, non-fibrillar, molded, collagen-based material suitable for ophthalmic uses, particularly for use in producing lenticular keratoprostheses, that are stronger (more suturable) than existing collagen materials. Simply repeating a typical single-stage crosslinking with increasing crosslinker concentrations either provides no improvement (as reported by Tardy) or doesn't produce an acceptable material (as shown by replication of Tardy). In contrast to prior art compositions and devices, Applicant's invention uses (i) diafiltered, lyophilized, redissolved and homogenized, non-fibrillar collagen, (ii) molds to form structured collagen hydrogels, (iii) high collagen concentration (137 mg/ml to 235 mg/ml), (iv) acidic pH (pH 3.7 to 5.5) in two crosslinking stages, and (v) very tight control of conductivity, temperature, and viscosity; Applicant's molded collagen material is ideal for use in producing ophthalmic devices, such as lenticular keratoprosthesis, and avoids the irregular mixing, unacceptable viscosity, and optical defects that plague single-stage crosslinking of high concentrations of collagen.

### SUMMARY OF THE INVENTION

The invention comprises a method of making molded, double-crosslinked (i.e., two stages of crosslinking), transparent, collagen materials, including “kPro lenticles”, and the collagen materials, ophthalmic devices, and kPro lenticles made by such method. The term “kPro lenticle” is used herein to mean a keratoprosthesis produced using the method of the invention. The shorter term, “kPro”, means a final product keratoprostheses made using only a single crosslinking stage. The term, “hydrogel blank”, means a post-first stage crosslinking, pre-second-stage crosslinking, intermediate product in the method of the invention. The terms “kPro” and “hydrogel blank” differ primarily in the fact that a kPro is a final product that is known in the prior art, while “hydrogel blank” is a precursor to the final product using the method of the invention (the final product being a “kPro lenticle”). The term “kPro lenticle” includes any other shape of final product produced using the method of the invention, e.g., use of a hemispheric, cubic, or other mold shape to product a final product. “Ophthalmic device” means a device that can be placed in a human or animal eye. If the ophthalmic device is implanted in the eye, e.g, by suturing a full thickness cornea of the invention in a human eye, the device is said to be “implantable”. “Implantable” means a device made of the collagen material of the invention is not rejected, extruded, immunogenic, or pathogenic, and is tolerated long-term after implantation. Collagen keratoprostheses made without toxic crosslinkers or rinses are known to be implantable, but most prior art collagen keratoprostheses are unacceptably weak or marginally acceptable. “Transparent” means collagen material and devices made with material have the property of transmitting 80% or higher percentage of incident light without appreciable scattering so that bodies lying beyond are seen clearly (a “transparent” material or device is “pellucid” or “optically clear”).

The method of the invention uses a first, low molar ratio, slower acting, crosslinker (the reaction of the first crosslinker with collagen in the reaction mixture is called a “first stage crosslinking”) to allow the reaction mixture to be injected into, and to conform to all surfaces of, a mold, typically a mold with a lenticular form. The form of a lenticular mold can be concave or convex, including variations in thickness and topology (e.g, toric) that provide refractive correction. The semi-crosslinked collagen is a hydrogel, is released from the mold as a “hydrogel blank”, and placed in a bath containing a higher concentration of the same or a different, “second” crosslinker. The action of the second crosslinker (the reaction of the second crosslinker with the semi-crosslinked collagen hydrogel blank is called a “second-stage crosslinking”) significantly increases structural strength (e.g., suture retention), without compromising the optical or morphological properties, of the kPro lenticle. Unlike the methods disclosed in Tardy and Chang, the method of the invention uses diafiltration, lyophilization, homogenization, and careful control of pH (monitored through a close surrogate, conductivity), temperature, and viscosity to substantially improve the strength and transparency of the collagen hydrogel such that the hydrogel can be used in implantable ophthalmic devices. Devices made with the collagen hydrogel of the invention far surpass the strength of previously known biopolymer hydrogels and equal the strength of some synthetic hydrogels. Synthetic hydrogels cannot be implanted in the eye for numerous reasons, e.g., corneal melt; poor glucose, metabolic product, and oxygen diffusivity; lack of re-innervation; and poor epithelial overgrowth (for corneal onlays).

Diafiltration is the preferred method of pH adjustment and of filtration based on molecular weight. Alternative methods to diafiltration, such as pH adjustment with base or acid, column filtration, or gel filtration, are either too time consuming for large volumes of collagen solution or result in pH surges and/or “pH ping-ponging”, i.e., overshooting the target pH of the collagen solution, which requires the addition of acid to correct too high pH values or of base to correct too low pH values. “pH ping-ponging” also undesirably dilutes the collagen solution concentration. Most importantly, pH adjustment of a collagen solution with NaOH produces a material that, after lyophilization, is insoluble in water, MES, and other common solvents suitable for use in producing implantable materials and devices.

The inventors discovered that diafiltration, and lyophilization are required to produce dry, uniform, collagen powder with greatly reduced small molecule contaminants. Collagen solutions prepared from such collagen powder have very precise, repeatable collagen concentrations and reaction properties that are essential to two-stage (and more generally, multi-stage) crosslinking methods.

The “reaction mixture” in the method of the invention is a mixture of homogenized collagen (Type I, II, III, IV or XI), preferably recombinant human collagen Type I or III, and a crosslinker, preferably EDC and NHS. The lyophilized collagen is dissolved to form a collagen solution, homogenized, and preferably centrifuged (to remove air bubbles) before being mixed with crosslinker. Removing air bubbles from homogenized collagen solutions is required for highest quality final products. For solutions of 4% or greater collagen concentration, centrifugation is highly preferred to remove air bubbles. Alternative methods for removing air bubbles, e.g., vacuum combined with ultrasound, typically produce lower quality final products compared to products produced using centrifugation of the homogenized collagen solution. After injection into a mold, the reaction mixture crosslinks, and the mass of crosslinked collagen released from the mold (a “hydrogel blank”) remains semi-crosslinked and very permeable. A second-stage crosslinking of the hydrogel blank is achieved by diffusing a higher molar ratio of a crosslinker, preferably EDC/NHS, into the hydrogel blank (i.e., the mass released from the mold). Diffusion of the second-stage crosslinker is typically by immersing hydrogel blanks in a bath of solvent and crosslinker. The low molar ratio crosslinker in the first stage facilitates the mixing and molding process because of the lower reaction rate (thus lower viscosity), and ensures the homogeneity of the hydrogel blank; the second-stage crosslinking of the hydrogel blank in a crosslinker solution (“second-stage bath”) fortifies the hydrogel blank by increasing the crosslinking density; the high permeability of the hydrogel blank to small molecule, chemical crosslinkers, such as EDC/NHS, enables a uniform, high crosslinking density in the final product, a kPro lenticle. The inventors theorize that omission of the diafiltration step permits “small molecule artifacts” to remain in the collagen; the small molecule artifacts interfere with the second-stage crosslinking.

Water for Injection (“WFI”) is the preferred type of water used to prepare the “aqueous solutions” described below. The mechanical performance data reported in Tables 2, 3, 7, 8, 12 and 13, which data are unexpectedly superior to prior art single-meshwork collagen materials, recite mean values; the mean values have acceptable standard deviations (which standard deviations are not recited in the Tables). The performance of the kPro lenticles can equal or surpass the performance of prior art interpenetrating polymer networks made with collagen.

Increased crosslinking density obtained using the method of the invention also improves in vivo kPro lenticle properties, especially collagenase resistance, optical properties, glucose diffusivity, water uptake, and durability. The two-stage crosslinking process of the invention also solves the problem of the reaction mixture crosslinking too rapidly and clogging supply channels in injection molding machines and other apparatus that dispense the reaction mixture into molds. The two-stage crosslinking process of the invention also affords better process control by eliminating mechanical weakness, optical defects, and/or conformational defects associated with single-stage crosslinking. The temperature, pH, viscosity, curing period, selected crosslinker and molarity, and other parameters of the two crosslinking stages enable fine tuning of manufacturing parameters (e.g., supply reservoir capacity, supply reservoir to mold distance) and final product characteristics, and produce much higher and consistent quality kPro lenticles compared with prior art methods.

In a preferred embodiment of the invention for replacement corneas, corneal inlays, corneal underlays, and corneal onlays, the method of the invention comprises: diafiltering a solution of collagen (including both commercially available solutions or solutions prepared from collagen powder); lyophilizing the diafiltered solution to produce a diafiltered, dry collagen powder; re-dissolving diafiltered, dry collagen powder in a solvent and homogenizing the solution; preferably centrifuging the collagen solution; mixing a low concentration of a crosslinker with the solution of homogenized collagen to form a reaction mixture; injecting the reaction mixture into a lenticular mold and allowing the collagen to crosslink in the mold to form a hydrogel blank; releasing the hydrogel blank from the mold; preferably quenching the crosslinking reaction and rinsing the hydrogel blank; placing the hydrogel blank in an aqueous bath containing a higher concentration of crosslinker, wherein the collagen in the hydrogel blank further crosslinks to form a lenticular keratoprosthesis, or “kPro lenticle”; and removing the kPro lenticle from the bath, quenching the crosslinking reaction, and rinsing (aka “washing”) the kPro lenticle.

### BRIEF DESCRIPTION OF THE DRAWINGS

FIG. 1. Light transmission of kPros (EDC/NHS/NH₂=0.4/0.4/1, RHCIII 12%, w/w) (Single-stage crosslinking)

FIG. 2. Light transmission of kPros (EDC/NHS/NH₂=0.4/0.4/1, RHCIII 23.5%, w/w) (Single-stage crosslinking).

FIG. 3. Light transmission of kPros (EDC/NHS/NH₂=0.7/0.7/1, RHCIII 23.5%, w/w) (Single-stage crosslinking).

FIG. 4. Light transmission of kPros (EDC/NHS/NH₂=1/1/1, RHCIII 23.5%, w/w) (Single-stage crosslinking).

FIG. 5. Effect of second-stage crosslinking on light transmission of hydrogel blanks and of kPro lenticles (EDC/NHS/NH₂=0.3/0.3/1, 20% RHCIII, w/w, 5% crosslinker, w/v, in second-stage bath) (comparison of single-stage and two-stage crosslinking).

FIG. 6. The effect of collagen (RHCIII) concentration on suture strength of kPros (EDC/NHS/NH₂=0.4/0.4/1) (single-stage crosslinking).

FIG. 7. The effect of crosslinker molar ratio on suture strength of kPros (collagen concentration RHCIII 23.5%, w/w) (single-stage crosslinking).

FIG. 8. Effect of second-stage crosslinking on suture strength of hydrogel blanks and kPro lenticles (collagen concentration RHCIII 20%, w/w, first stage crosslinker molar ratio EDC/NHS/NH₂=0.3/0.3/1, the percentages in the right margin represent the crosslinker concentration %, w/v, in the second-stage bath).

FIG. 9. Effect of RHCIII collagen concentration on mechanical properties of kPros (single-stage crosslinking, molar ratio EDC/NHS/NH₂=0.4/0.4/1).

FIG. 10. Effect of crosslinker molar ratio on mechanical properties of kPros (single-stage crosslinking, collagen concentration RHCIII 23.5%, w/w).

FIG. 11. Effect of second-stage-crosslinking on mechanical properties of collagen flatsheet (collagen concentration RHCIII 20%, w/w, first stage crosslinker molar ratio EDC/NHS/NH₂=0.3/0.3/1, the percentages in the right margin represent the crosslinker concentration %, w/v, in the second-stage crosslinking bath).

FIG. 12. Denaturing temperature vs. RHCIII collagen concentration (single-stage crosslinking, EDC/NHS/NH₂=0.4/0.4/1).

FIG. 13. Denaturing temperature vs. crosslinker molar ratio (single-stage crosslinking, collagen concentration RHCIII 23.5%, w/w).

FIG. 14. Denaturing temperature vs. second-stage crosslinking crosslinker concentration (collagen concentration RHCIII 20%, w/w, the percentages in right margin represent the crosslinker concentration %, w/v, in the second-stage crosslinking bath).

FIG. 15. Dynamics of the second-stage crosslinking (collagen concentration RHCIII 20%, w/w, first stage crosslinker molar ratio EDC/NHS/NH₂=0.3/0.3/1, 1% crosslinker, w/v, in the second-stage crosslinking).

FIG. 16. Denaturing temperature for different pH buffer solution in two-stage crosslinking (collagen concentration RHCIII 20%, w/w, first stage crosslinking molar ratio EDC/NHS/NH₂=0.3/0.3/1, 1% crosslinker, w/v, in the second-stage crosslinking).

FIG. 17. Denaturing temperature of kPros for different incubation time in pre-crosslinking (collagen concentration RHCIII 20%, w/w, first stage crosslinking molar ratio EDC/NHS/NH₂=0.3/0.3/1, no second-stage bath, A: incubation at room temperature for 12 hours and then at 37° C. for 24 hours, B: incubation at room temperature for 12 hours).

FIG. 18. Denaturing temperature of second-stage crosslinked kPro lenticles for different incubation time in first stage crosslinking (collagen concentration RHCIII 20%, w/w, first stage crosslinking molar ratio EDC/NHS/NH₂=0.3/0.3/1, 1% crosslinker, w/v, second-stage crosslinking bath, A: incubation at room temperature for 12 hours and at 37° C. for 24 hours, B: incubation at room temperature for 12 hours).

FIG. 19. The percent residual mass of the kPros and kPro lenticles vs. collagenase incubation time (in hours) using collagenase assay (BEC-808/RHCIII prototype: collagen concentration RHCIII 12%, w/w, single-stage crosslinking EDC/NHS/NH₂—0.4/0.4/1; F-1: collagen concentration RHCIII 20%, w/w, single-stage crosslinking EDC/NHS/NH₂—0.3/0.3/1; F-4: collagen concentration RHCIII 20%, w/w, single-stage crosslinking EDC/NHS/NH₂—0.3/0.3/1, 5% crosslinker, w/v, second-stage crosslinking bath).

FIG. 20. Flowchart for processing of collagen for KPro lenticles, part one.

FIG. 21. Flowchart for processing of collagen for KPro lenticles, part two.

### DETAILED DESCRIPTION OF THE PREFERRED EMBODIMENTS

In one preferred method of the invention, a first, low molar ratio, slower acting, crosslinker is mixed with diafiltered, lyophilized, re-dissolved, and homogenized collagen, preferably recombinant human collagen (“RHC”) Type I or III to form a reaction mixture; the reaction mixture is injected into a mold cavity (“mold”) in an injection molding machine. For replacement corneas, corneal onlays, corneal underlays, and corneal inlays, the mold cavity is lenticular. “Diafiltration” uses ultrafiltration membranes to remove microsolutes from a solution, preferably by tangential flow filtration (“TFF”). In tangential flow filtration, the fluid is pumped tangentially along the surface of the membrane. An applied pressure serves to force a portion of the fluid through the membrane to the filtrate side. Tangential flow filtration is preferable to “normal flow filtration” (aka “dead end filtration”) since TFF produced better experimental results. Small molecules are separated from a solution while retaining larger molecules in the retentate. Essentially, continuous diafiltration used herein is a technique in which buffer salts and/or acids from solutions containing 0.2-0.5% (w/v) collagen, preferably RHC with a concentration of 0.25-0.35% (w/v), are washed with water, preferably water for injection (“WFI”), wherein the concentration and volume remain unchanged since WFI is added as filtrate is removed. The temperature of the WFI during diafiltration is kept constant at 10° C.-22° C., preferably 12° C.-18° C. The initial pH of the collagen solution is typically in the range of pH 1.9 to 2.4, with a corresponding conductivity of 3800-2500 micro Siemens per centimeter (“μS/cm”). Inline monitoring of the filtrate is preferably used to avoid contamination of the retentate. The conductivity measurement of the filtrate is an indirect measurement of the pH of the retentate. The diafiltration process continues until a target pH for the retentate is reached, preferably a filtrate pH in the range of pH 3.7-4.5, and filtrate conductivity in the range of 43-165 μS/cm are reached. As the conductivity value approaches the target conductivity, it is preferred to suspend the diafiltration process and determine the pH of a sample removed from the retentate. Even though conductivity and pH of collagen solutions are related, conductivity values are much more stable and easier to monitor than pH given the low ionic strength of the protein solution in the late stages of diafiltration, which makes accurate, direct measurement of pH very difficult. If concentration and conductivity values for collagen solution are not accurately achieved, the crosslinking methods and desired properties (optical, mechanical, thermal stability, and collagenase degradation, etc.) of the kPros lenticles described below will be negatively impacted, resulting in a less than desirable final product.

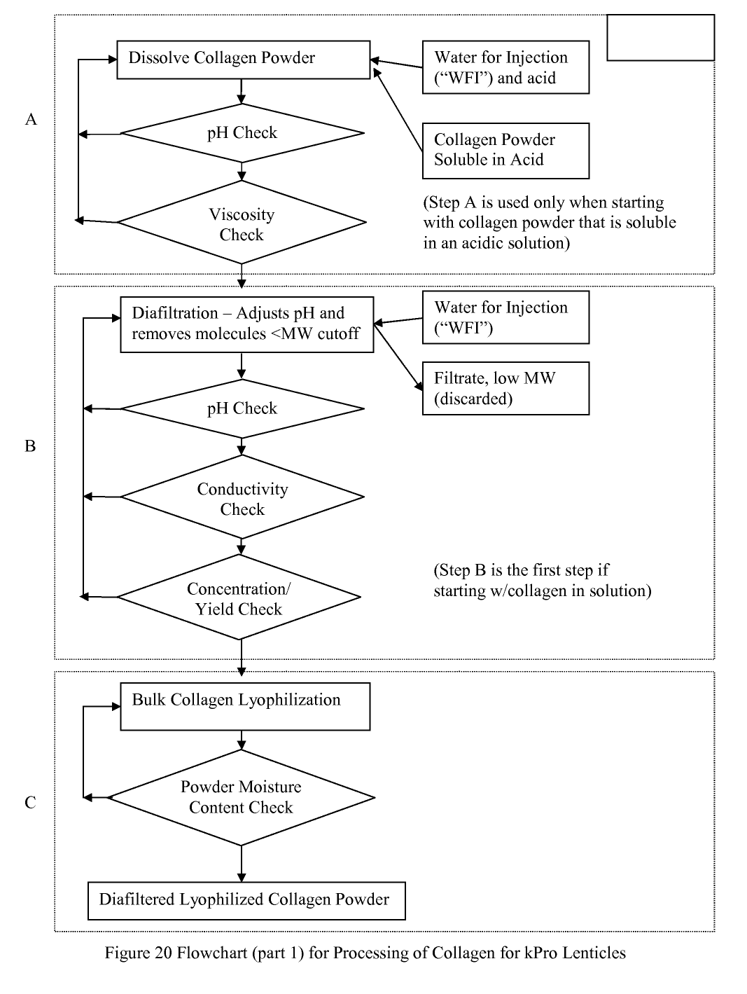

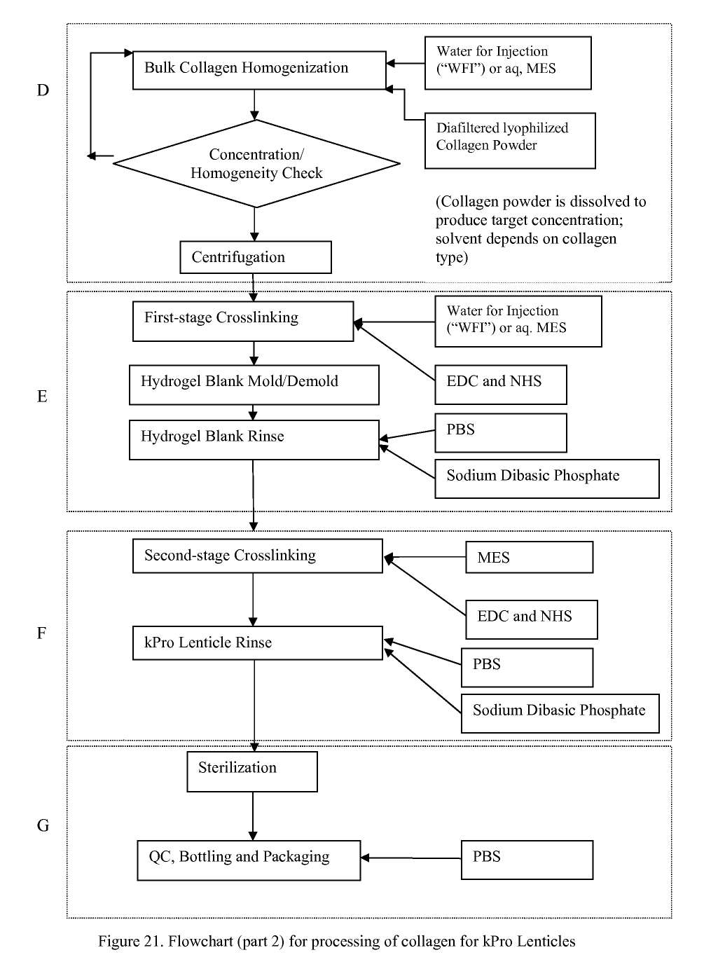

As shown in FIGS. 20-21, Step A is used only when starting with collagen powder that is soluble in an acidic solution; in Step A, the collagen powder is dissolved so that the solution can be diafiltered. In Step A, acidic powdered collagen, preferably RHC Type I or III, is dissolved to target concentration, preferably 0.25-0.35% (w/v), and viscosity, 3000-6000 centiPoise (“cP”), preferably 4500-5500 cP. Pre-cooled WFI at a temperature of 2° C.-10° C., preferably 4° C.-6° C., is preferably used to dissolve powdered collagen when the collagen is lyophilized from acidic solution; the collagen solution produced is acidic. WFI, or if needed an aqueous acid commonly used in pharmaceutical production (such as aqueous HCl), is used in Step A to prepare an aqueous collagen solution in the range of pH 1.0 to 3.0, preferably in the range of pH 1.9-2.4. Viscosity and pH are checked before step B. In Step B, collagen solution (either from Step A or sourced as an acidic collagen solution from a supplier), preferably RHC Type I or III, is diafiltered with pre-cooled WFI, preferably at a temperature of 2° C.-10° C., even more preferably 4° C.-6° C., to a target conductivity with corresponding pH, preferably 43-165 μS/cm and pH 3.7-4.5, and viscosity of 300-900 cP, preferably 650-850 cP. Conductivity of filtrate is monitored and checked until the target conductivity is reached. Concentration, yield, and pH of retentate are checked before proceeding to Step C. In Step C, the solution is then lyophilized to a powder with powder moisture content check until the water content of the powder is typically not greater than 21%. In Step D, the lyophilized collagen powder is dissolved in either pre-cooled WFI or 0.5M MES, preferably at a temperature of 2-10° C., even more preferably at 4° C.-6° C., and homogenized to a predetermined concentration of 3.0-23.5% (w/w), preferably 12%-15% (w/w); the concentration and homogeneity are checked, and if acceptable, the solution is preferably centrifuged to remove air bubbles. Temperature is tightly controlled during Step D; homogenization is conducted preferably within a temperature range of 4° C.-20° C., even more preferably from 4° C.-10° C., and centrifugation is conducted preferably within a temperature range of 4° C.-10° C., even more preferably from 4° C.-6° C. In Step E, the collagen solution from step D is mixed with crosslinker, preferably EDC and NHS, in aqueous solution (WFI or 0.5M MES) to form a reaction mixture, preferably within a temperature range of 0° C.-10° C., even more preferably from 4° C.-6° C. The reaction mixture is immediately injected into a mold; while the reaction mixture is in the mold, the crosslinker causes crosslinks to form among and within collagen molecules (first stage crosslinking); after a selected time (“incubation time”) in the mold, the reaction mixture in a given mold becomes a “hydrogel blank”. The hydrogel blanks are released from the mold (“demolded”). The demolded hydrogel blanks are preferably immersed (or rinsed) in quencher, preferably sodium dibasic phosphate (Na₂HPO₄) in WFI, to quench the first-stage crosslinking reaction, then rinsed with buffer (preferably, PBS). In Step F, the hydrogel blanks from Step E are placed in a solution (“bath”) of stronger concentration crosslinker in 0.5M MES, preferably within a temperature range of 20° C.-25° C., even more preferably from 21° C.-23° C., where a second-stage of crosslinking occurs as the crosslinker in the bath diffuses into the hydrogel blanks to cause the formation of more crosslinks After a selected incubation time and temperature in the bath, the hydrogel blanks become kPro lenticles, and are removed from the bath, the crosslinking reaction quenched, preferably with Na₂HPO₄ in WFI, and rinsed, preferably in PBS. If the second-stage crosslinking reaction is not quenched, and the kPro lenticles rinsed, the risk of cytotoxicity or immunogenicity after implantation increases (unpublished data). Rinsing of the kPro lenticles is preferably done in two steps: a first rinse within a temperature range of 20° C.-25° C., even more preferably from 21° C.-23° C., and then a second rinse within a temperature range of 4° C.-10°, even more preferably from 4° C.-6° C. In Step G, if the kPro lenticles are not immediately sterilized and implanted, the kPro lenticles are sterilized and packaged in a buffer solution. 0.5M MES is recited through this Description as the buffered aqueous solution used when mixing collagen powder in buffered aqueous solution, and when exposing a hydrogel blank to a second crosslinker (second-stage crosslinking) Buffers other than MES (such as HCl, 3-(N-morpholino) propanesulfonic acid (“MOPS”), etc.), and concentrations other than 0.5M (such as 0.1M to 1.0M), can be used to formulate a buffered aqueous solution or bath, but 0.5M MES is preferred as the buffered aqueous solution or buffered aqueous bath. Similarly, Na₂HPO₄ in WFI is the preferred quencher, but other quenchers may be used; phosphate buffered saline (“PBS”) is the preferred buffer for rinsing and storage, but other buffers may be used.

Injection molding machines used to produce kPros, as well as the kPro lenticles of the invention, typically do not use a moveable platen or high compression forces, although the feed stock (reaction mixture) is pressurized. Typically, multiple molds are contained in a single tooling. After injection, first stage crosslinking creates a moderately crosslinked hydrogel blank within the mold cavity. The hydrogel blank is semi-crosslinked (i.e., significantly less than all amine groups in each collagen polymer are linked to amine groups in the same or other collagen polymers in the hydrogel blank) and very permeable, and has desired optical and morphological properties as specified by the three-dimensional design of the mold cavity, choice of collagen Type, and injection conditions (typically, pH 3.7-5.5, room temperature, atmospheric pressure, 0.5-3 minutes injection period). After first stage crosslinking (reaction mixture left in-mold at room temperature typically for 8-12 hours, 100% relative humidity), the hydrogel blanks are removed from the molds, rinsed and transferred to a bath at pH in a range of 3.7 to 5.5, and comprising 0.5M 2-(N-morpholino) ethanesulfonic acid (“MES”), sterile water, and a second, higher concentration crosslinker. The second crosslinker diffuses from the bath solution into each partially solidified, very permeable hydrogel blank; the second crosslinker causes second-stage crosslinking within each hydrogel blank (typically at room temperature, atmospheric pressure, 6-12 hours, 100% relative humidity above bath). Second-stage crosslinking creates a flexible, hydrogel kPro lenticle with significantly increased structural strength in all axes compared with the hydrogel blank, but does not compromise the optical or morphological properties of the hydrogel blank.

The same or different crosslinker, preferably EDC/NHS, can be used as the first and second crosslinker, so long as the molarity of the first crosslinker is in a range of lower molarity, and below a threshold value, compared to the molarity of the second crosslinker. When the first stage crosslinker is EDC/NHS, crosslinker molarity should be below 0.5 EDC, 0.5 NHS, and 1.0 NH₂, (reaction mixture molarities are hereafter abbreviated, e.g., the preceding EDC/NHS/NH₂ molarities are hereafter abbreviated 0.5/0.5/1), but not lower than 0.1/0.1/1. Values below the minimum can be used, but the reaction rate is typically too slow for commercial purposes, or the hydrogel blank typically may not be strong enough for demolding and to maintain its shape during the second-stage crosslinking Further increasing the molar ratio of the crosslinkers in the first stage above 0.5/0.5/1 increases the crosslinking reaction rate, making the crosslinking process too fast, thus resulting in difficulties in mixing the reaction mixture as well as in injection of the reaction mixture into the molds. When the second crosslinker is also EDC/NHS, reaction mixture molarity should be below 500/500/1 (equivalent to 10% crosslinker in the bath), but more than 5/5/1 (5/5/1 is equivalent to 0.1% crosslinker in the bath).

The low molar ratio crosslinker in the first stage provides a lower reaction rate (and thus lowers the mixture viscosity), ensures the homogeneity of the hydrogel, and facilitates the mixing and supply of the reaction mixture from a mixing reservoir to the mold cavities. second-stageDuring the second-stage crosslinking, the crosslinker in the bath easily diffuses into the hydrogel blanks to create the higher crosslinking density that characterizes the kPro lenticles, but without the tight control of collagen concentration, pH, viscosity, homogeneity, and temperature described above, optical and mechanical properties of a kPro lenticle will be suboptical or unacceptable.

A kPro lenticle produced using the method of the invention can be the full thickness of the stroma (aka “full thickness cornea” or “replacement cornea”), or less than full thickness in the case of a corneal onlay (a device that is placed posterior to any remaining corneal epithelium and Bowman's layer and anterior to the existing or remaining stroma), a corneal inlay (a device that is placed within stroma of the cornea), or a corneal underlay (a device that is placed posterior to the existing stroma and anterior to Descemet's membrane and the corneal endothelium). A kPro lenticle produced using the method of the invention also provides improved mechanical properties, e.g., suture resistance, and improves properties of the kPro lenticle “in-situ” (after placement in the eye), such as collagenase resistance, optical properties, glucose and gas diffusivity, water uptake, and durability.

Formulations with different collagen Types and concentrations, first stage molar ratios of EDC/NHS/NH₂ reaction mixture, and second-stage molar ratios of EDC/NHS/NH₂ reaction mixture were studied, as shown in the Examples below. The preferred reaction mixture formulation and process described in the Examples produced kPro lenticles with superior optical properties (light transmission and refractive index), and significantly improved mechanical properties (tensile and suture strength), thermal stability, and collagenase resistance. The method of the invention provides a desirable and unexpected increase of crosslinking density in the hydrogel blanks as a result of diafiltration, lyophilization, homogenization, tight control of pH, temperature, and viscosity, and the second-stage crosslinking A preferred method of the invention uses the same crosslinker in both crosslinking stages, but in different molarities.

The novel steps of the invention, i.e., diafiltration, lyophilization, and homogenization before first crosslinking, and the associated tight control of pH, viscosity, temperature, and collagen concentration, can also be used to produce improved collagen-based interpenetrating polymer networks. The method of the invention can also be adapted to have three or more stages of crosslinking, as opposed to only two-stages, by using increments of crosslinker concentration per stage.

Generally, process steps of the method of the invention can be conducted within a temperature range of 4° C. to 26° C., except the temperature range in first-stage crosslinking can be 0° C. to 10° C. Superior optical and mechanical properties of final product are achieved, however if the temperature range of the process steps is controlled as follows: during diafiltration, within a range of 10° C. to 22°, preferably within a range of 12° C. to 18° C.; during homogenization, within a range of 4° C. to 20°, preferably within a range of 4° C. to 10° C.; during centrifugation, within a range of 4° C. to 20°, preferably within a range of 4° C. to 6° C.; during first stage crosslinking, within a range of 0° C. to 10°, preferably within a range of 4° C. to 6° C.; during second-stage crosslinking, within a range of 20° C. to 25°, preferably within a range of 23° C. to 25° C.; and during rinsing, within a range of 20° C. to 25°, preferably within a range of 23° C. to 25° C.

The collagen material produced using the methods of the invention is a hydrogel and can be used not only as an ophthalmic device, but also as a tissue scaffold (a support that maintains tissue contour), drug delivery device (a time-release substance and route (e.g., oral, parenteral, implanted) by which a therapeutic agent is administered), wound dressing (aka wound healing agent and/or carrier of one or more wound healing agents), or other collagen hydrogel based devices. The collagen hydrogels produced by the methods of the invention can be desiccated for storage and distribution.

In Vitro Cytotoxicity Test.

In vitro cytotoxicity testing was performed on collagen materials made using the method of the invention to evaluate whether the kPro lenticles had an overt toxic effect on primary human corneal epithelial cells. Molded kPro lenticles, such as those made in Examples 1 to 3 below, were each cut into three pieces to achieve replicate analyses. Each replicate was placed in a well of a 48-well tissue culture plate (BD Falcon Cat. No. 08-772-1C). Commercially available human corneal epithelial cells (Invitrogen, Cat. No. C-018-5C) in logarithmic phase growth were plated at 50,000 cells/well onto the pieces of kPro lenticles and incubated at 37° C. The control wells contained only growth medium (Invitrogen, Cat. No. 17005042). At 24 hours post-plating, cultures were examined under microscope at 40× and 100× power to determine the extent of cell adhesion onto the collagen material and cell morphology. Cells were observed daily over the course of up to one week for visible signs of toxicity, such as changes in cell size or morphology, and qualitative assessment of proliferation (cells reaching confluence). The collagen material of the invention was found to be non-cytotoxic.

### EXAMPLES

### Materials

Human type I collagen (VitroCol, Human Collagen type I (HCl), Advanced Biomatrix, Cat. No. #5007-A, San Diego, Calif.)
Recombinant Human Collagen type I (RHCI) (Recombinant Human Collagen Type I, Cat. No. W1019, CollPlant, Israel)
Recombinant Human Collagen type III (RHCIII) (Recombinant Human Collagen Type III, FibroGen, Cat. No. rhC3-012, San Francisco, Calif.)
“EDC”, 1-ethyl-3′-(3-dimethylaminopropyl)carbodiimide hydrochloride. (Sigma-Aldrich, Cat. No. E1769, St. Louis, Mo.)
“NHS”, N-hydroxy-succinimide (Sigma-Aldrich, Cat. No. 130672, St. Louis, Mo.)
Phosphate Buffered Saline (“PBS”) with magnesium and calcium (aka Dulbecco's) (Invitrogen Corp. (Gibco), Cat. No. 14040-117, Carlsbad, Calif.)
“WFI” (Water for Injection) (sterile Water for Injection, Fisher Scientific (Hyclone), Cat. No. SH3022125, Logan, Utah)
Collagenase with Clostridium Histolyticum Type IA EC 3.424.3 or equivalent (“collagenase”) (Clostridium Histolyticum EC 3.424.3 or equivalent, Sigma-Aldrich, Cat. No. C9891, St. Louis, Mo.)
“MES”, 2-(N-morpholino)ethanesulfonic acid (Sigma-Aldrich, Cat. No. 76039, St. Louis, Mo.)
Sodium dibasic phosphate Na₂HPO₄ (Sigma-Aldrich, Cat. No. 57907, St. Louis, Mo.)
Contact lens molds with mirror finish (Makrolon Molds, Quickparts, Cat. No. MS-419.5TL, Atlanta, Ga.)
Pellicon 2 Cassette Ultracel regenerated cellulose ultrafiltration membrane (“PLCHK”), (Millipore, Cat. No. P2C100001, Billerica, Mass.)
Legato 380 dual syringe pump system (KD Scientific, Holliston, Mass.)

### Example 1

### Recombinant Human Collagen Type III (“RHCIII”)

### Methods

Diafiltration.

0.25-0.35% (w/v) RHCIII solution is diafiltered using Millipore Pellicon holder (EMDMillipore, Billerica, Mass.) and PLCHK ultrafiltration membrane (as shown in FIG. 20, Step B). Viscosity of RHCIII is between 3000-6000 centiPoise (“cP”), preferably 4500-5500 cP. As pre-cooled WFI buffer (pH 7.0) is pumped into the retentate at a set flow rate, the salt/acid containing filtrate is removed at an equivalent rate. In-line conductivity and pH is monitored until the conductivity of filtrate typically equals 43 μS/cm with corresponding retenate, a diafiltered RHCIII (“DRHCIII”) solution, having a pH of 4.0-4.5 with viscosity between 300-900 cP, preferably 650-850 cP.

Lyophilization.

0.15-0.2% (w/v) DRHCIII is then lyophilized using a VirTis Advange Plus (VirTis, Gardiner, N.Y.) bulk tray lyophilizer between 30-60 hours as shown in FIG. 20, Step C. The small variation in concentration of DRHCIII arises from rinsing the retentate container with a barely adequate amount of WFI and adding the rinsed retentate to DRHC to minimize material loss.

Homogenization.

Lyophilized DRHCIII is dissolved in WFI to a collagen concentration of 3%-23.5% (w/w), preferably 12-15% (w/w), and then homogenized in a Legato 380 dual syringe pump system as shown in FIG. 21, Step D. Target viscosity of the collagen solution is between 3000-90,000 cP, preferably 5000-50,000 cP. All high-capacity homogenizers, other than dual syringe pump designs, tested by the inventors were rejected based on dead zones, material loss, and/or poor homogeneity of output, although it is possible that some designs other than dual syringe pumps may work adequately.

Collagen Gel Preparation.

Collagen gel was prepared using EDC and NHS at three variations of an EDC-to-NHS-to-collagen primary amine group molar ratio, namely, 0.1/0.1/1, 0.2/0.2/1 and 0.3/0.3/1 (first-stage crosslinking, as shown in Step E, FIG. 21), followed by a second-stage crosslinking process (Step F, FIG. 21). Briefly, in the first stage, 100 to 1000 mg aliquots of 3-23.5% (w/w), preferably 12%-15% (w/w), collagen were loaded into a syringe mixing system, and calculated volumes of EDC (10%, w/v) and NHS (10%, w/v) in WFI solutions were added and mixed to form the reaction mixture. The reaction mixture was dispensed into contact lens molds and cured with 100% relative humidity (pH 4.0-4.5, room temperature, 8-12 hours). Reaction injection molding (RIM) machines, such as the Graco PD44 metering valve with controller, and reservoir tanks, are available from Graco (Graco, Inc., Minneapolis, Minn.) and dual pump syringe systems, such as the KD Scientific Legato 380 Emulsifier (KD Scientific, Inc., Holliston, Mass.) were used for initial molding. Lenticular mold cavities are available from Quickparts (Quickparts, Cat. No. MS-419.5TL, Atlanta, Ga.) and lenticular holding clamps from Prototypes Plus (Prototypes Plus, Menlo Park, Calif.). It is preferable to quench the first-stage crosslinking reaction with Na₂HPO₄ in WFI before rinsing the hydrogel blanks with PBS. Quenching and PBS rinsing remove residual crosslinkers. In the second stage, the clear, partially crosslinked, cornea-shaped, hydrogel blanks were immersed in a 1 to 10% (w/v) EDC and NHS aqueous solution containing 0.5M MES (“second-stage bath”) at room temperature to effect a second-stage crosslinking reaction (pH 3.7.-4.5, 6-12 hours, 100% relative humidity above bath). After rinsing with Na₂HPO₄ to quench the second-stage crosslinking reaction and rinsing with PBS, the kPro lenticles were stored in PBS for characterization. Data showing the effect of the recited ranges of reagents is presented in the Experimental Results and Tables 1, 2, 3 and 4 below.

Characterization.

The same characterization apparatus and characterization methods were used in all Examples.

Light Transmission.

A spectrophotometer was used to determine the light transmission of the kPro lenticles within visible light wavelengths (400-700 nm). Three locations of the kPro lenticles were examined (12 o'clock, 3 o'clock orientations, and lifting the spectroscopic cuvette 1 mm up), and raising and rotating the cuvette tests for optical uniformity of the kPro lenticles. Light transmission above 80% is considered “transparent”.

Refractive Index.

The refractive index of kPro lenticles was determined using a Reichert Abbe refractometer (Grainger, Chicago, Ill.). The refractive index of kPro lenticles described in the Examples ranged from 1.36 to 1.38, which is acceptable for ophthalmic devices.

Water Uptake.

The water content of the kPro lenticles was determined by lyophilizing the kPro lenticles (each, a “sample”) and weighing each sample before and after lyophilization.

Suture Pull Retention.

Suturability of kPro lenticles was evaluated by double suture pull method using 10-0 nylon sutures on an Instron Stress/Strain tester (Instron, Norwood, Mass.).

Mechanical Test.

kPro lenticles were tested for ultimate tensile strength (“UTS”), ultimate elongation at break (“UTE”), elastic modulus (“EM”), and energy at break (“ETB”) on an Instron Stress/Strain tester (Instron, Norwood, Mass.).

Differential Scanning Calorimetry (“DSC”).

The thermal denaturing temperature (T_{d}) of collagen was determined using a computerized DSC system (DSCi Series, Instrument Specialists Inc., Twin Lakes, Wis.) to assess the effectiveness of the collagen crosslinking reaction.

Collagenase Assay.

The in vitro collagenase resistance of the kPro lenticles was determined by monitoring the residual mass percentage of the gel incubated in a 1 mg/mL collagenase solution as a function of time. Collagenase from Clostridium histolyticum, Type IA is available from Sigma-Aldrich, Cat. No. C9891 (St. Louis, Mo.).

Glucose Diffusivity.

PermeGear Valia-Chien cells (PermeGear Inc., Hellertown, Pa.) were used to determine the glucose diffusivity of the kPro lenticles. In the test system, glucose diffuses from a donor chamber through one kPro lenticle to a receptor chamber. Water jackets keep the temperature of the cells constant and the stirring bars keep the concentration in both chambers uniform all the time. Periodically, solution samples are drawn from the receptor chamber and the concentrations are measured using the glucose assay kit. The change of concentration of the solutes with time is used to calculate the diffusivity of the solutes through the kPro lenticles membrane.

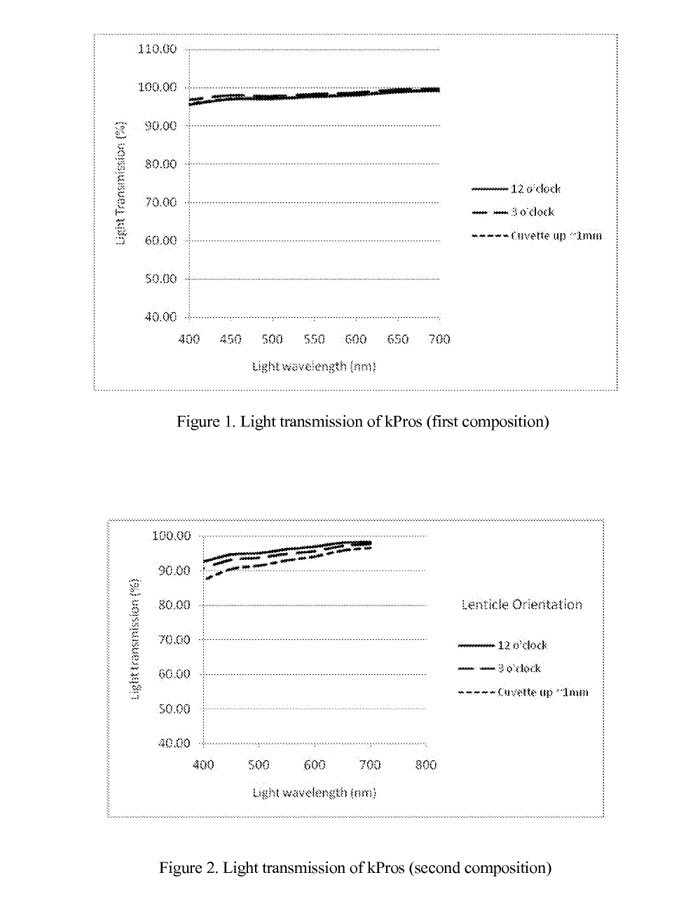

As shown in FIG. 1, at an RCHIII collagen concentration of 12% (w/w) in WFI and a molar ratio of EDC/NHS/NH₂=0.4/0.4/1, clear kPros were made by thorough mixing of reaction mixture (single-stage crosslinking; no second-stage crosslinking or bath). Light transmission within visible wavelengths was above 80%, relative standard deviation of light transmission at different locations of the kPros was below 3%, indicating homogeneity of the kPros.

As shown in FIG. 2, at higher collagen concentration (23.5%, w/w), mostly transparent kPros were made by mixing of reaction mixture (EDC/NHS/NH₂=0.4/0.4/1, RHCIII 23.5% (w/w) in WFI, single-stage crosslinking; no second-stage crosslinking or bath). Light transmission within visible wavelengths was above 80%, however, relative standard deviation of light transmission at different locations of the kPros was above 3% indicating non-homogeneity of the kPros. Due to the higher viscosity and faster crosslinking reaction at higher collagen concentration (23.5%, w/w), reduced mixing times were used before injection into molds. Thorough mixing of the reaction mixture after addition of crosslinker solution to RHCIII was impossible due to the fast crosslinking reaction; the solution would have solidified before injection with normal mixing times.

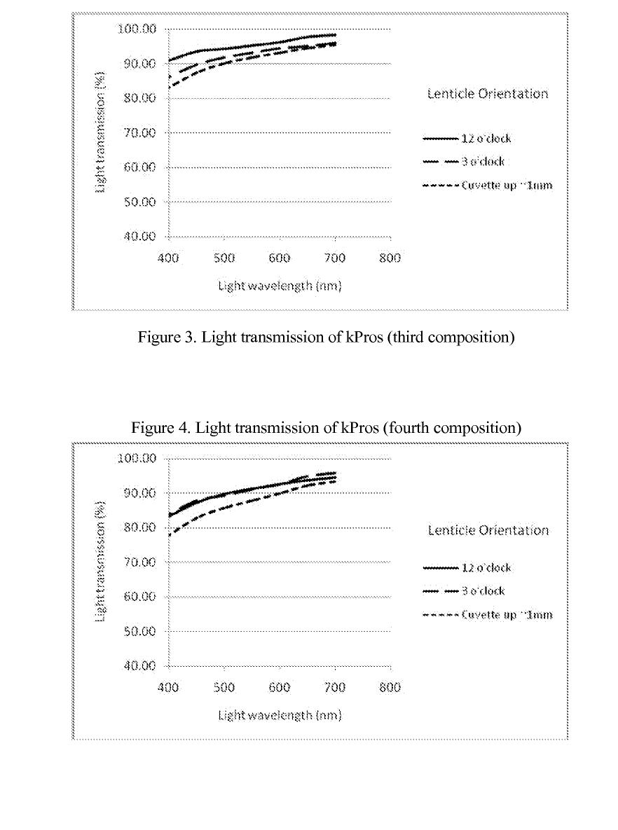

As shown in FIG. 3, mostly transparent kPros (which also had white cloudy areas) at higher collagen concentration and crosslinker molar ratio were made by mixing of reaction mixture (EDC/NHS/NH₂=0.7/0.7/1, RHCIII 23.5% (w/w) in WFI single-stage crosslinking; no second-stage crosslinking or bath). Light transmission within visible wavelengths was above 80%, relative standard deviation of light transmission at different locations of the kPros was above 3%, which indicates non-homogeneity of the kPros, probably due to insufficient mixing (reduced mixing) after EDC/NHS addition because of the rapid crosslinking reaction (FIG. 3).

As shown in FIG. 4, mostly transparent kPros (with white cloudy areas) were made by mixing of reaction mixture (EDC/NHS/NH2=1/1/1, RHCIII 23.5% (w/w) in WFI, single-stage crosslinking; no second-stage crosslinking or bath). Light transmission within visible wavelengths was above 80%, relative standard deviation of light transmission at different locations of the kPros was above 3%, which indicates non-homogeneity of the kPros, probably due to insufficient mixing after EDC addition because of the rapid crosslinking reaction under the conditions as indicated in FIG. 4.

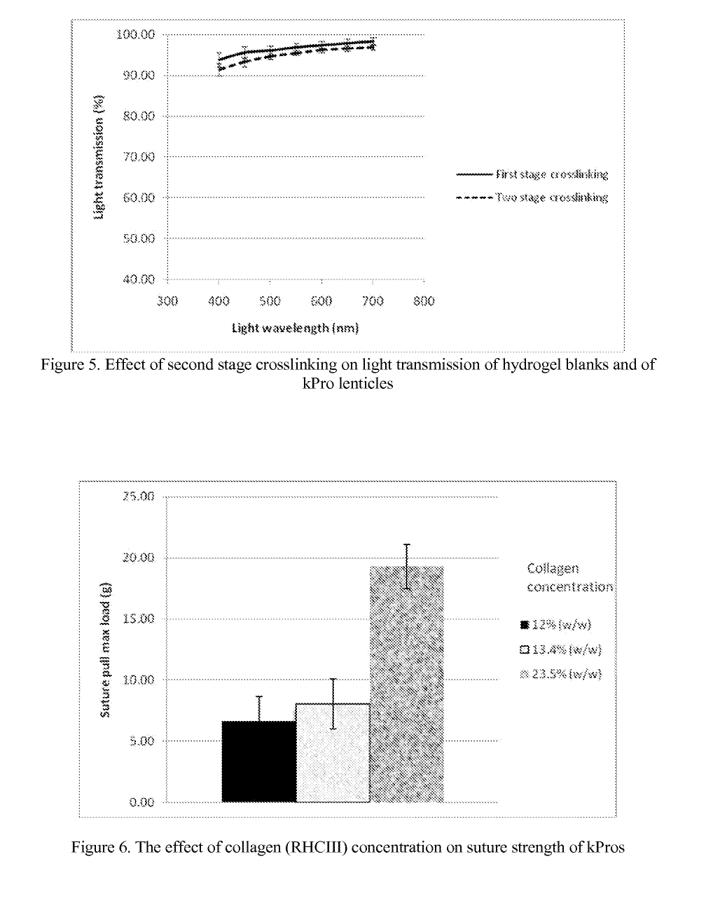

As shown in FIG. 5, at 20% collagen concentration (EDC/NHS/NH₂=0.3/0.3/1, 5% crosslinker, (w/v)), two crosslinking stages only slightly decreased the light transmission of the kPro lenticles; however, the optical properties of the two-stage crosslinked kPro lenticles met the criteria generally accepted as required for keratoprostheses. The error bars indicated the variance of light transmission at different kPro lenticle locations.

As shown in FIG. 6, as collagen concentration increased from 12% to 23.5% (w/w), initial collagen concentration before single-stage crosslinking, EDC/NHS/NH₂=0.4/0.4/1), the suture strength of the kPros increased about two fold. The results are the average of three samples. The final collagen concentrations after crosslinking were 9%, 10%, and 17%, respectively. The decrease of collagen concentrations in the final kPro lenticles was due to the dilution from crosslinker solutions.

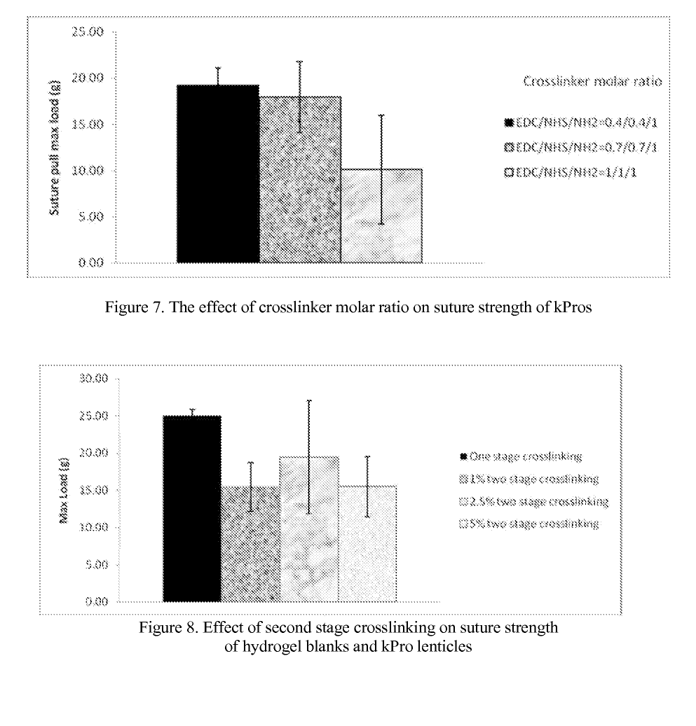

As shown in FIG. 7, further increase of crosslinker molar ratio from 0.4 to 0.7 to 1.0 at collagen concentration of 23.5% (w/w) did not improve the suture strength of the kPros, probably due to the insufficient mixing and single-stage crosslinking. The bigger error bars at higher molar ratios also indicated the heterogeneity of the kPros due to inadequate mixing.

FIG. 8 shows the results of two-stages of crosslinking The suture strength of the kPro lenticles slightly decreased, compared with kPros made by single-stage crosslinking, at a collagen concentration of 20% (w/w) (FIG. 8) because the kPro lenticles became more brittle after the second-stage crosslinking (see below section for mechanical properties of the materials).

Mechanical Tensile Strength.

Consistent with suture strength results, ultimate strength and elastic modulus increased significantly with increasing collagen concentration. The lowest ultimate elongation at 23.5% (w/w) concentration indicated the stiffness was highest at this concentration.

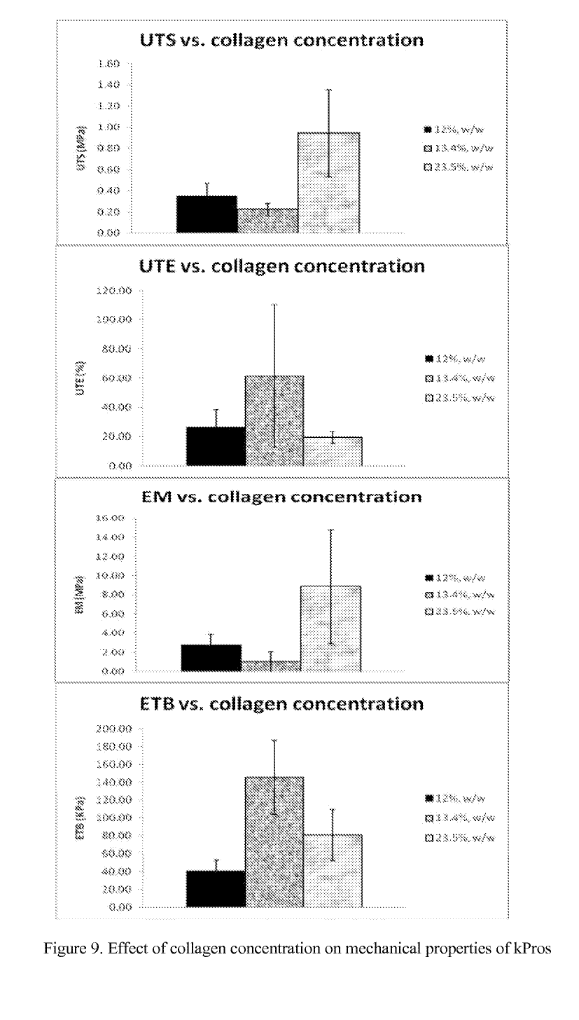

FIG. 9 shows that the mechanical strength of kPros improved significantly with increasing collagen concentration, using single-stage crosslinking.

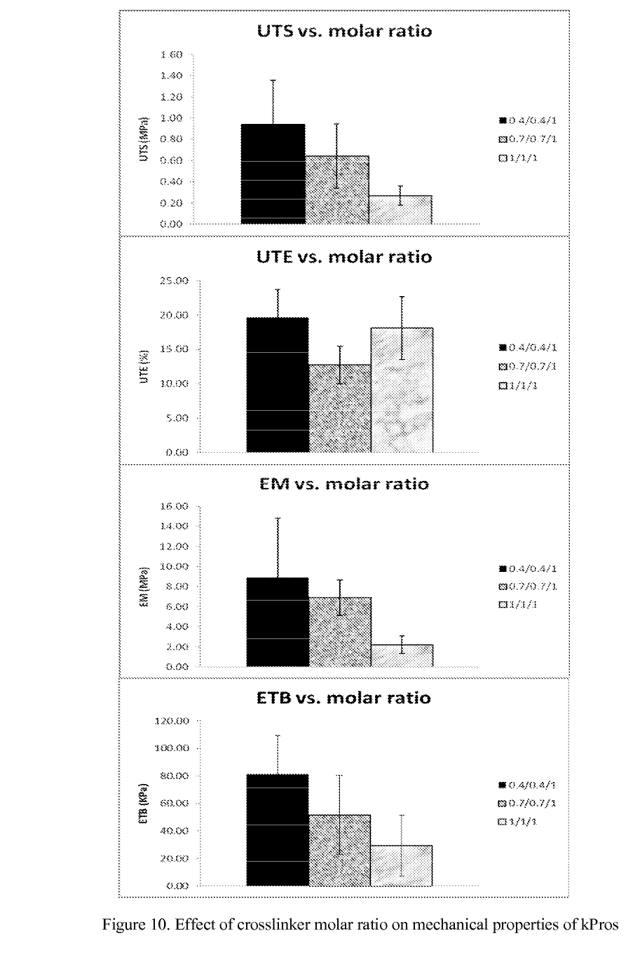

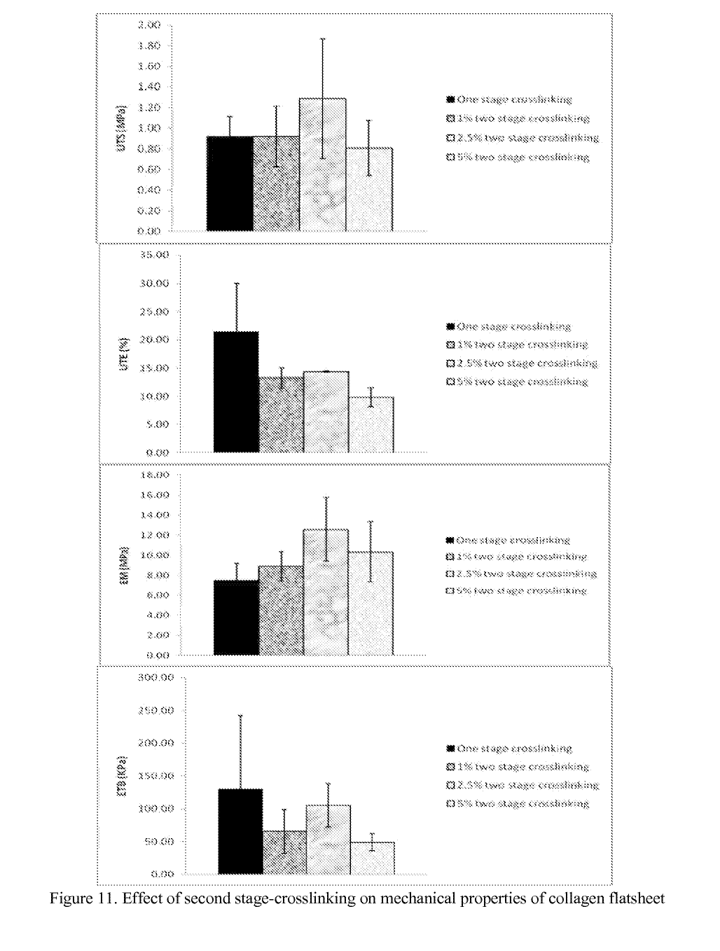

As shown in FIG. 10, further increase of crosslinker molar ratio in the single-stage crosslinking did not improve mechanical properties of kPros. In fact, probably due to the poor mixing of the materials, the mechanical properties (ultimate tensile strength (“UTS”), elastic modulus (“EM”), ultimate elongation (“UTE”) and energy to break (“ETB”) deteriorated at higher molar ratios.

As shown in FIG. 11, two-stage crosslinking increased brittleness of the material of hydrogel blanks and of kPro lenticles as indicated by increased EM and decreased UTE. The toughness of the material also decreased after second-stage crosslinking (decreased ETB). However, the ultimate strength (UTS) of the materials increased at 2.5% second-stage-crosslinking and did not change significantly at other crosslinker concentrations due to second-stage-crosslinking.

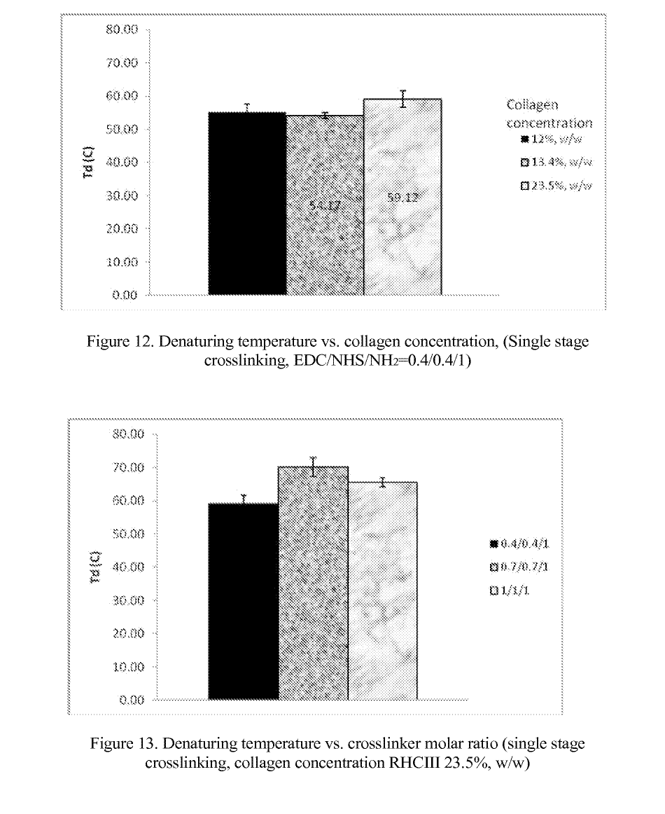

As shown in FIG. 12, the denaturing temperatures of the EDC/NHS single-stage crosslinked collagen increased from 45° C. (raw collagen) to 55° C. at 12% (w/w) collagen concentration, and up to 59° C. at 23.5% (w/w) collagen cencentration. It is well known that chemical crosslinking of proteins (e.g., collagen) improves the thermal properties of the materials, e.g., produces elevated denaturing temperatures. The increase in temperature of denaturation reflects higher crosslinking density at higher collagen concentration.

As shown in FIG. 13, at high collagen concentration (23.5%, w/w), the crosslinking density reached the maximal point at EDC/NHS/NH₂ of 0.7/0.7/1. However, due to the insufficient mixing, multiple denaturing peaks (data not shown) were found at higher molar ratios (including 0.7/0.7/1 and 1/1/1). We hypothesize that crosslinker was not homogeneously distributed in the kPros so that different parts were crosslinked in different degrees (resulting in multiple denaturing peaks in DSC thermal graphs, data not shown).

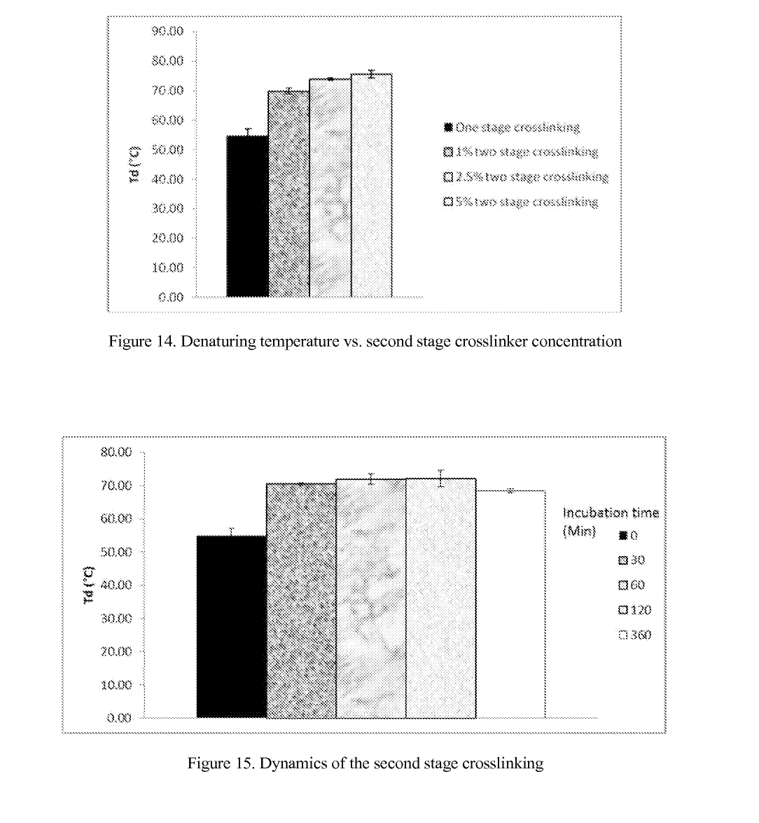

As shown in FIG. 14, the crosslinking density (indicated by denaturing temperature and enthalpy) of the kPro lenticles increased almost linearly with increasing crosslinker concentration during the second-stage crosslinking.

As shown in FIG. 15, the dynamic/fine-tuning experiment shows that the second-stage-crosslinking reaction was fast and reached equilibrium within 30 minutes.

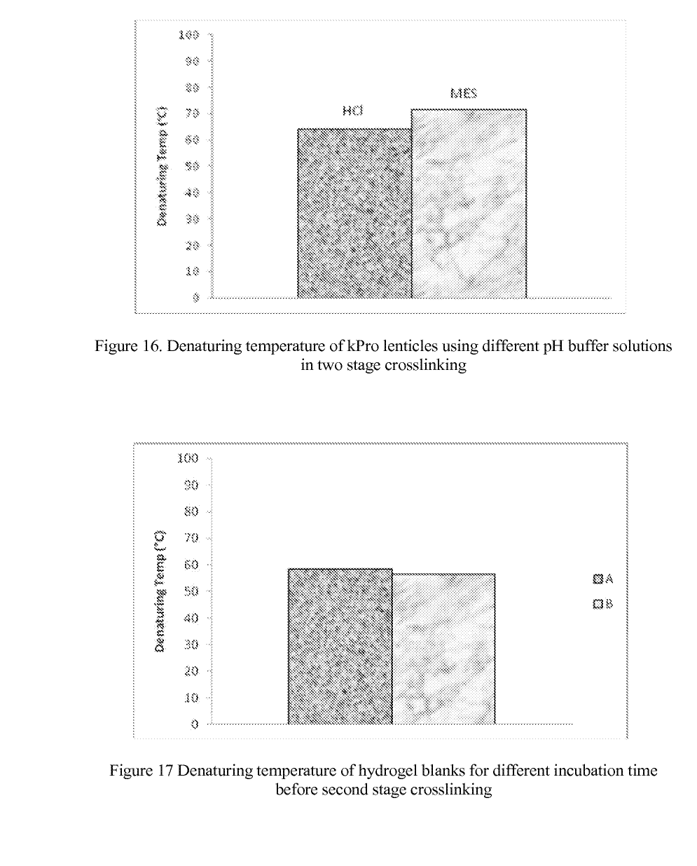

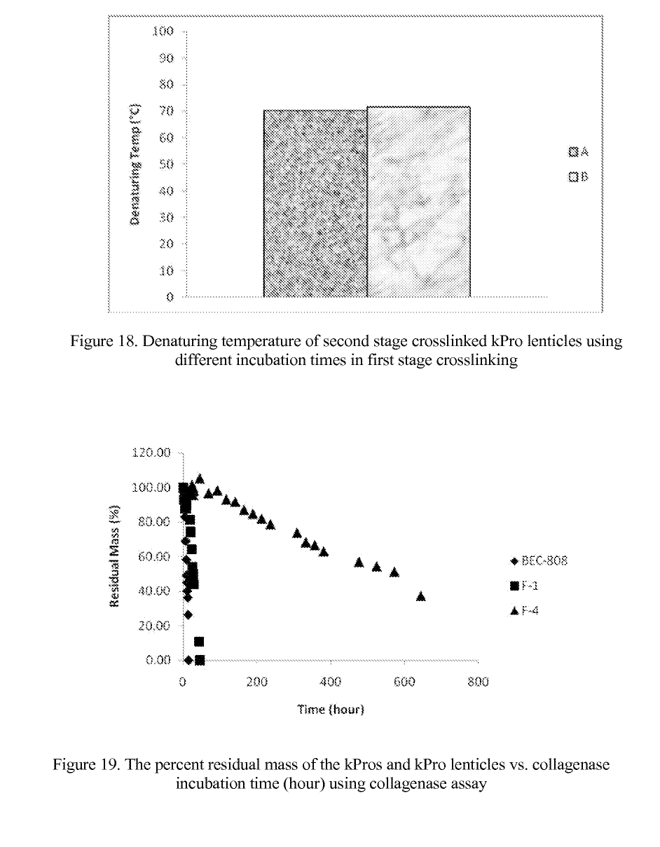

As shown in FIGS. 16, 17, and 18, a manufacturing optimization study indicated the necessity of using MES instead of HCl for pH adjustment/buffering for the second-stage-crosslinking reaction (FIG. 16). MES was found to be a better buffering reagent; the pH of the reaction mixture drifted to lower values when using HCl compared to using MES (data not shown). The second-stage aqueous bath that includes MES, HCl, or other buffering agent is referred to as a “buffered aqueous bath”. In the first stage crosslinking, incubation of the hydrogel blanks at room temperature for 12 hours showed slightly lower denaturing temperature, indicating that incubation at 37° C. for 24 hours may slightly increase the thermostability of the hydrogel blanks (FIG. 17) compared with incubation at room temperature; however, in the second-stage crosslinking, this effect was masked and showed almost identical thermostability in both cases (FIG. 18).

Collagenase Resistance.

Tables 4 show the results of collagenase assays using collagenase (In Table 4, 8.3 CDU/ml were used, where “CDU” means “collagen digestion unit”) and PBS with Ca²⁺ and Mg²⁺. Two-stage crosslinking significantly increased the collagenase resistance of the kPro lenticles. The kPro lenticles with two-stage crosslinking (F-2, F-3, and F-4) did not degrade for 92 hours while single-stage crosslinked kPros (BEC-808/RHCIII prototype) degraded within 20 hours under the same conditions. These results correlate well with the DSC results.

Table 1 outlines the formulations studied using RHCIII. Table 2, 3 and 4 tabulate the key properties of the formulations studied.

### Example 2

### VitroCol, Human Collagen type I (“HCI”)

### Methods

Diafiltration.

0.25-0.35% (w/v) HCI solution is diafiltered using Millipore Pellicon holder (EMDMillipore, Billerica, Mass.) and PLCHK ultrafiltration membrane (as shown in FIG. 20, Step B). Target viscosity of HCl is between 3000-6000 centiPoise (“cP”), preferably 4500-5500 cP. As WFI buffer is pumped into the retentate at a set flow rate, the salt/acid containing filtrate is removed at an equivalent rate. In-line conductivity and pH is monitored until the conductivity of filtrate equals 55 μS/cm, which corresponds to a retentate, DHCI solution, pH of 4.0-4.5 with viscosity between 300-900 cP, preferably 650-850 cP. As outlined in Table 9, different target conductivity values were explored to ensure that the most desirable physical, mechanical, optical, thermal and permeable properties of the kPro lenticles were achieved.

Lyophilization.

0.15-0.2% (w/v) diafiltered HCl (“DHCI”) is then lyophilized using a VirTis Advange Plus (VirTis, Gardiner, N.Y.) bulk tray lyophilizer between 30-60 hours (Step C, FIG. 20). The small variation in concentration of DHCI arises from rinsing the retentate container with a barely adequate amount of WFI and adding the rinsed retentate to DHCI to minimize material loss.

Homogenization.

Lyophilized DHCI is dissolved in WFI to 3-23.5% (w/w), preferably 12%-15% (w/w) and then homogenized in a Legato 380 dual syringe pump system (Step D, FIG. 21). Target viscosity of the collagen solution is between 3,000-90,000 cP, preferably 5,000-50,000 cP.

Collagen Gel Preparation.

Collagen gel was prepared using EDC and NHS at three variations of an EDC-to-NHS-to-collagen primary amine group molar ratio, namely, 0.3/0.3/1, 0.4/0.4/1 and 0.5/0.5/1 (first stage, Step E, FIG. 21), followed by a second-stage crosslinking process (Step F, FIG. 21). Briefly, in the first stage, 100 to 1000 mg aliquots of 3-23.5% (w/w) HCI collagen, preferably wild type human collagen Type I, 12%-15% (w/w), were loaded into a syringe mixing system and calculated volumes of EDC (10%, w/v) and NHS (10%, w/v) in WFI solutions were added and mixed with the reaction mixture. The reaction mixture was dispensed into contact lens molds and cured with 100% relative humidity (pH 4.0-4.5, room temperature, 8-12 hours). Lenticular mold cavities are available from Quickparts (Quickparts, Cat. No. MS-419.5TL, Atlanta, Ga.) and lenticular holding clamps from Prototypes Plus (Prototypes Plus, Menlo Park, Calif.). After Na₂HPO₄ reaction quenching (quenching is preferred, but not required after the first-stage crosslinking) and PBS rinsing to remove residual cross linkers, in the second stage, the clear, partially crosslinked, cornea-shaped, hydrogel blanks were immersed in a 0.2 to 1.0% (w/v) EDC and NHS aqueous solution containing 0.5M MES (“second-stage bath”) at room temperature to effect a second-stage crosslinking reaction (pH 3.7-4.5, 6-12 hours, 100% relative humidity above bath). After rinsing with Na₂HPO₄ in WFI to quench the second-stage crosslinking reaction and rinsing with PBS, the kPro lenticles were stored in PBS for characterization as described in sections 0059 to 0067.

Table 5 tabulates the formulations of HCl-based, double-crosslinked collagen material produced with different crosslinker concentrations.

Tables 6-9 tabulate the key properties of the formulations studied. The mechanical properties, suturability and collagenase resistance increased when compared with a single-stage crosslinked RHCIII prototype formulation, while the optical properties and solute (like glucose) permeability were retained.

### Example 3

### Recombinant Human Collagen Type I (“RHCI”)

### Methods

Diafiltration.

0.25-0.35% (w/v) RHCI solution is diafiltered using Millipore Pellicon holder (EMDMillipore, Billerica, Mass.) and PLCHK ultrafiltration membrane (as shown in FIG. 20, Step B). Target viscosity of RHCI is between 3000-6000 centiPoise (“cP”), preferably 4500-5500 cP. As WFI buffer is pumped into the retentate at a set flow rate, the salt/acid containing filtrate is removed at an equivalent rate. In-line conductivity and pH is monitored until the conductivity of filtrate equals 165 μS/cm, which corresponds to a g retentate, DRHCI solution, pH of 4.0-4.5 with viscosity between 300-900 cP, preferably 650-850 cP. As outlined in Table 14, different target conductivity values were explored to ensure that the most desirable physical, mechanical, optical, thermal and permeable properties of the kPro lenticles were achieved. Trial and error was needed to definitively correlate both filtrate conductivity and retentate pH values.

Lyophilization.

0.015-0.02% (w/v) diafiltered RCHI (“DRHCI”) is then lyophilized using a VirTis Advange Plus (VirTis, Gardiner, N.Y.) bulk tray lyophilizer between 30-60 hours as shown in FIG. 20, Step C. The small variation in concentration of DRHCI arises from rinsing the retentate container with a barely adequate amount of WFI and adding the rinsed retentate to DRHCI to minimize material loss.

Homogenization.

Lyophilized DRHCI is dissolved in WFI or MES (depending on the formulation) to 3-23.5% (w/w), preferably 12%-15% (w/w) and then homogenized in a Legato 380 dual syringe pump system (Step D, FIG. 21). Target viscosity of the collagen solution is between 3,000-90,000 cP, preferably 5,000-50,000 cP.

Collagen Gel Preparation.

Collagen gel was prepared using EDC and NHS at three variations of an EDC-to-NHS-to-collagen primary amine group molar ratio, namely, 0.3/0.3/1, 0.4/0.4/1 and 0.5/0.5/1 (first stage, Step E, FIG. 21), followed by a second-stage crosslinking process (Step F, FIG. 21). Briefly, in the first stage, 100 to 1000 mg aliquots of 3-23.5% (w/w) RHCI collagen, preferably 12%-15% (w/w) were loaded into a syringe mixing system and calculated volumes of EDC (10%, w/v) and NHS (10%, w/v) in 0.5M MES solutions were added and mixed to form the reaction mixture. The reaction mixture was dispensed into contact lens molds and cured with 100% relative humidity (pH 3.7-4.0, room temperature, 8-12 hours). Lenticular mold cavities are available from Quickparts (Quickparts, Cat. No. MS-419.5TL, Atlanta, Ga.) and lenticular holding clamps from Prototypes Plus (Prototypes Plus, Menlo Park, Calif.). After PBS rinsing to remove residual cross linkers, in the second-stage, the clear, partially crosslinked, cornea-shaped, hydrogel blanks were immersed in a 0.1 to 1.0% (w/v) EDC and NHS aqueous solution containing 0.5M MES (“second-stage bath”) at room temperature to effect a second-stage crosslinking reaction (pH 3.7.-4.5, 6-12 hours, 100% relative humidity above bath). After rinsing with Na₂HPO₄ in WFI to quench the reaction, the kPro lenticles were rinsed with PBS and stored in PBS for characterization as described in sections 0059 to 0067.

Table 10 tabulates the formulations of RHCI studied. Formulation 2 was different from the other formulations in the list of RHCI formulations; in formulation 2, collagen powder was dissolved in WFI, not in MES, as outlined in Table 14. All the other formulations utilized 0.5M MES as the buffer to dissolve the collagen powder and to make the crosslinker solutions in the first stage.

Tables 11-14 the key properties of the formulations studied in Example 3. When WFI was used to dissolve RHCI powder and to make the crosslinker solutions in the first stage, the resulted lenticles showed poorer light transmission than the formulations with MES. The mechanical properties, suturability and collagenase resistance increased when compared with single-stage crosslinked RHCIII prototype formulation, while the optical properties and solute (like glucose) permeability were retained. All the tested lenticles were found to be non-cytotoxic.

### REFERENCES

Angele, P., J. Abke, R. Kujat, H. Faltermeier, D. Schumann, M. Nerlich, B. Kinner, C. Englert, Z. Ruszczak, R. Mehrl, and R. Mueller. 2004. Influence of different collagen species on physico-chemical properties of crosslinked collagen matrices. Biomaterials 25: 2831-2841.
Chen, R., H. Ho, and M. Sheu. 2005. Characterization of collagen matrices crosslinked using microbial transglutaminase. Biomaterials 26: 4229-4235.
Daamen, W. F., H. T. B. van Moerkerk, T. Hafmans, L. Buttafoco, A. A. Poot, J. H. Veerkamp, and T. H. van Kuppevelt. 2003. Preparation and evaluation of molecularly-defined collagen-elastin-glycosaminoglycan scaffolds for tissue engineering. Biomaterials 24: 4001-4009.
Duan, X., C. McLaughlin, M. Griffith, and H. Sheardown. 2007. Biofunctionalization of collagen for improved biological response: scaffolds for corneal tissue engineering. Biomaterials 28: 78-88.
Duan, X., and H. Sheardown. 2005. Crosslinking of collagen with dendrimers. J. Biomed. Mater. Res. 75A: 510-518.
Duan, X., and H. Sheardown. 2006. Dendrimer crosslinked collagen as a corneal tissue engineering scaffold: mechanical properties and corneal epithelial cell interactions. Biomaterials 27: 4608-4617.
Duan, X., and H. Sheardown. 2007. Crosslinking of collagen with riboflavin and UVA for corneal repair and partial thickness artificial cornea. Private communication.
Griffith, M, Carlsson, D J, Li, F, Liu, Y, and Rafat, M. Ophthalmic devices and related methods and compositions, US Patent Application 20080269119
Hanthamorongwit, M., W. H. Reid, and M. H. Grant. 1996. Chondroitin-6-sulphate incorporated into collagen gels for the growth of human keratinocytes: the effect of cross-linking agents and diamines. Biomaterials 17: 775-780.
Hey, K. B., C. M. Lachs, M. J. Raxworthy, and E. I. Wood. 1990. Crosslinked fibrous collagen for the use as a dermal implant: control of the cytotoxic effects of glutaraldehyde and dimethylsuberimidate. Biotech Appl Biochem 12: 85-93.
Kohlhaas, M. 2008. Collagen crosslinking with riboflavin and UVA-light in keratoconus. Ophthalmologe 105: 785-96.
Lee, C. R., A. J. Grodzinsky, and M. Spector. 2001. The effects of cross-linking of collagen glycosaminoglycan scaffolds on compressive stiffness, chrodrocyte-mediated contraction, proliferation and biosynthesis. Biomaterials 22: 3145-3154.
Li, F., D. Carlsson, C. Lohmann, E. Suuronen, S. Vascotto, K. Kobuch, H. Sheardown, R. Munger, M. Nakamura, and M. Griffith. 2003. Cellular and nerve regeneration within a biosynthetic extracellular matrix for corneal transplantation. Proc Natl Acad Sci USA 100: 15346-51.
Liu, Y., L. Gan, D. J. Carlsson, P. Fagerholm, N. Lagali, M. A. Watsky, and a. et. 2006. A simple cross-linked collagen tissue substitute for corneal implantation. IOVS 47: 1869-1875.
McKegney, M. T., I.; Grant, M. H. 2001. THe influence of crosslinking agents and diamines on the pore size, morphology and the biological stability of collagen sponges and their effect on cell penetration through the sponge matrix. J. Mat. Sci. Mat. Med. 12: 833-844.
Nam, Kwangwoo, T. Kimura, A. Kishida. 2007. Physical and biological properties of collagen-phospholipid polymer hybrid gels. Biomaterials 28: 3153-3162.
Olde Damink, L. H. H. D., P. J.; van Luyn, M. J. A.; van Wachem, P. B.; Nieuwnehuis, P.; Feijen, J. 1996. Cross-linking of dermal sheep collagen using a water-soluble carbodiimide. Biomaterials 17: 765-773.
Osborne, C. S. B., J. C.; Smith, D.; Savakis, M.; Grant, M. H. 1998. Investigation into the tensile properties of collagen/chondroitin-6-sulphate gels: the effect of crosslinking agents and diamines. Med. Biol. Eng. Comput. 36: 129-134.
Park, S. P., J.; Kim, H. O.; Song, M. J.; Suh, H. 2002. Characterization of porous collagen/hyaluronic acid scaffold modified by 1-ethyl-3-(3-dimethylaminopropyl)carbodiimide cross-linking Biomaterials 23: 1205-1212.
Pieper, J. S. H., T.; Veerkamp, J. H.; van Kuppevelt, T. H. 2000. Development of tailor-made collagen-glycosaminoglycan matrices: EDC/NHS crosslinking, and ultrastructural aspects. Biomaterials 21: 581-593.
Pieper, J. S. O., A.; Dijkstra, P. J.; Veerkamp, J. H.; van Kuppevelt, T. H. 1999. Preparation and characterization of porous crosslinked collagenous matrices containing bioactive chondroitin sulphate. Biomaterials 20: 847-858.
Pieper, J. S. v. d. K., P. M.; Hafmans, T.; Kamp, J.; Bumac, P.; van Susante, J. L. C.; van den Berg, W. B.; Veerkamp, J. H.; van Kuppevelt, T.H.2002. Crosslinked type II collagen matrices: preparation, characterization, and potential for cartilage engineering. Biomaterials 23: 3183-3192.
Rafat, M., F. Li, P. Fagerholm, N. S. Lagali, M. A. Watsky, R. Munger, T. Matsuura, and M. Griffith. 2008. PEG-stabilized carbodiimide crosslinked collagen-chitosan hydrogels for corneal tissue engineering. Biomaterials 29: 3960-72.
Rault, I. F., V.; Herbage, D. 1996. Evaluation of different chemical methods for crosslinking collagen gel, films and sponges. J. Mat. Sci. Mat. Med. 7: 215-221.
Speer, D. C., M.; Eskelton, C.; Ulreich, J. 1980. Biological effects of residual glutaraldehyde-tanned collagen biomaterials. J. Biomed. Mater. Res. 14: 753-764.
Zeeman, R., P. J. Dijkstra, P. B. van Wachem, M. J. A. van Luyn, M. Hendriks, P. T. Cahalan, and J. Feijen. 1999. Successive epoxy and carbodiimide crosslinking of dermal sheep collagen. Biomaterials 20: 921-931.

TABLE 1
Summary of formulations - FibroGen Recombinant Human
Collagen Type III (“RHCIII”)

| | Collagen | | Two-stage |
|---|---|---|---|
| | Concentration | | crosslinking |
| Formulation # | (w/w %) | EDC/NHS/NH2 | (w/v %) |
| 1 | 20 | 0.3/0.3/1 | 0 |
| 2 | 20 | 0.3/0.3/1 | 1 |
| 3 | 20 | 0.3/0.3/1 | 2.5 |
| 4 | 20 | 0.3/0.3/1 | 5 |
| 5 | 20 | 0.2/0.2/1 | 5 |
| 6 | 15 | 0.4/0.4/1 | 1 |
| 7 | 23.5 | 0.4/0.4/1 | 0 |
| 8 | 23.5 | 0.4/0.4/1 | 10 |
| 10 | 23.5 | 0.4/0.16/1 | 0 |
| 11 | 23.5 | 0.7/0.7/1 | 0 |
| 12 | 23.5 | 1/1/1 | 0 |

TABLE 2
Summary of results of key properties of formulations studied -
FibroGen Recombinant Human Collagen Type III (“RHCIII”)

| | Water | Transmission | Tensile Strength |
|---|---|---|---|
| Formulation # | Content (%) | (400-700 nm) | (Mpa) |
| 1 | 84.4 ± 0.6 | 96.6% | 0.92 ± 0.20* |
| 2 | 85.4 ± 0.4 | 94.0%** | 0.92 ± 0.29* |
| 3 | 84.5 ± 0.5 | 93.3%** | 1.28 ± 0.58* |
| 4 | 84.0 ± 0.7 | 95.0% | 0.81 ± 0.27* |
| 5 | ND | 95.6% | 0.36 ± 0.07 |
| 6 | ND | 97.6% | 0.19 ± 0.04 |
| 7 | ND | 94.3% | 0.95 ± 0.41 |
| 8 | ND | 86.3% | 0.57 ± 0.29 |
| 10 | ND | 81.9% | 0.26 ± 0.06 |
| 11 | ND | 65.9% | 0.64 ± 0.30 |
| 12 | ND | 81.9% | 0.27 ± 0.09 |
| Human | 80 | >85% | 3.8 |
| Cornea | | | |
| RHC-III | 90 | 97.5% | 0.35 ± 0.12 |
| Prototype | | | |

*Values from strips cut from flat sheets
**dehydrated/rehydrated lenticles
ND: Not determined

TABLE 3
Summary of results of key properties of formulations studied - FibroGen
Recombinant Human Collagen Type III (“RHCIII”)

| Formu- | | Elastic | | |
|---|---|---|---|---|
| lation | Elongation at | Modulus | Suture | |
| # | break (%) | (Mpa) | Strength (g) | T_{d} (° C.) |
| 1 | 21.42 ± 8.45* | 7.51 ± 1.66* | 25.00 ± 5.86 | 54.78 ± 2.30 |
| 2 | 13.28 ± 1.83* | 8.92 ± 1.47* | 15.46 ± 3.25 | 69.84 ± 1.03 |
| 3 | 14.4 ± 20.11* | 12.59 ± 3.19* | 19.44 ± 7.58 | 73.77 ± 0.32 |
| 4 | 9.88 ± 1.68* | 10.36 ± 3.01* | 15.51 ± 4.04 | 75.55 ± 1.35 |
| 5 | 7.26 ± 1.08 | 7.26 ± 0.03 | 15.58 ± 5.89 | 76.25 ± 0.61 |
| 6 | 9.84 ± 1.87 | 2.44 ± 0.62 | 6.80 ± 2.90 | 67.63 ± 0.95 |
| 7 | 19.61 ± 4.12 | 8.85 ± 5.95 | 19.29 ± 1.82 | 59.12 ± 2.50 |
| 8 | 7.84 ± 1.52 | 10.13 ± 5.22 | 21.12 ± 6.00 | 77.48 ± 0.41 |
| 10 | 25.01 ± 3.78 | 1.78 ± 0.04 | 16.22 ± 4.03 | ND |
| 11 | 12.74 ± 2.72 | 6.92 ± 1.78 | 17.99 ± 3.84 | 70.08 ± 2.80 |
| 12 | 18.12 ± 4.59 | 2.23 ± 0.87 | 10.10 ± 5.87 | 65.53 ± 1.44 |
| Human | 60 ± 15 | 3-13 | >75.6 | 65.1 |
| Cornea | | | | |
| RHC-III | 26.62 ± 12.02 | 2.82 ± 1.06 | 6.65 ± 2.00 | 55.21 ± 2.52 |
| Proto- | | | | |
| type | | | | |

*Values from strips cut from flat sheets
ND: Not determined

TABLE 4
Summary of results of key properties of formulations studied -
FibroGen Recombinant Human Collagen Type III (“RHCIII”)

| | | | Diafiltration |
|---|---|---|---|
| Formulation | Glucose Perm | Collagenase | Conductivity |
| # | (cm²/s) | (hours) | Value |
| 1 | 2.96 × 10⁻⁶ | 45 | 43 μS/cm |
| 2 | 3.70 × 10⁻⁶ | >92 | 43 μS/cm |
| 3 | 3.33 × 10⁻⁶ | >92 | 43 μS/cm |
| 4 | 3.33 × 10⁻⁶ | >644 | 43 μS/cm |
| 5 | ND | ND | 43 μS/cm |
| 6 | 3.70 × 10⁻⁶ | ND | 43 μS/cm |
| 7 | ND | ND | 43 μS/cm |
| 8 | ND | ND | 43 μS/cm |
| 10 | ND | ND | 43 μS/cm |
| 11 | ND | ND | 43 μS/cm |
| 12 | ND | ND | 43 μS/cm |
| Human | 2.5 × 10⁻⁶ | 624 | N/A |
| Cornea | | | |
| RHC-III | 2.96 × 10⁻⁶ | 14 | 43 μS/cm |
| Prototype | | | |

* Values from strips cut from flat sheets
ND: Not determined

TABLE 5
Summary of Formulations - VitroCol Human Collagen Type I

| | Collagen | | Two-stage |
|---|---|---|---|
| | Concentration | | Crosslinking |
| Formulation # | (w/w %) | EDC/NHS/NH₂ | (w/v %) |
| 1 | 12% | 0.4/0.4/1 | 0.5% |
| 2 | 12% | 0.4/0.4/1 | 0.2% |

TABLE 6
Summary of results of key properties of formulations studied -
VitroCol Human Collagen Type I

| | Light | | |
|---|---|---|---|
| Formulation | Transmission | Refractive | Td |
| # | (400 nm-700 nm) | Index | (° C.) |
| 1 | 93.3% | 1.3635 | 66.69 |
| 2 | 95.3% | 1.3657 | 61.40 |
| Human Cornea* | >85% | 1.3760 | 65.10 |
| RHCIII | 97.5% | 1.3500 | 55.21 |
| Prototype | | | |

*Literature Values

TABLE 7
Summary of results of key properties of formulations studied -
VitroCol Human Collagen Type I

| | Tensile Strength | Elongation at | Elastic Modulus |
|---|---|---|---|
| Formulation # | (MPa) | break (%) | (MPa) |
| 1 | 1.06 | 15.27 | 11.79 |
| 2 | 1.13 | 22.22 | 8.08 |
| Human | 3.8 | 60 ± 15 | 3-13 |
| Cornea* | | | |
| RHCIII | 0.35 | 26.62 | 2.82 |
| Prototype | | | |

*Literature Values
ND: Not Determined

TABLE 8
Summary of results of key properties of formulations studied -
VitroCol Human Collagen Type I

| | Energy at Break | Suture | |
|---|---|---|---|
| | (Toughness) | Retention | Glucose Perm |
| Formulation # | (KPa) | (g) | (cm²/s) |
| 1 | 81 | 12.00 | 2.06 × 10⁻⁶ |
| 2 | 76 | 16.41 | 2.59 × 10⁻⁶ |
| Human Cornea* | N/A | >75.6 | 2.5 × 10⁻⁶ |
| RHCIII | ND | 6.65 | 2.96 × 10⁻⁶ |
| Prototype | | | |

*Literature Values
ND: Not Determined

TABLE 9
Summary of results of key properties of formulations studied -
VitroCol Human Collagen Type I

| | Diafiltration | Water | Collagenase | |
|---|---|---|---|---|
| | Conductivity | Content | Resistance | |
| Formulation # | Value | (%) | (hours) | Cytotoxicity |
| 1 | 43 μS/cm | 87.6 | 24 | Non-Cytotoxic |
| 2 | 55 μS/cm | 88.4 | 14 | Non-Cytotoxic |
| Human Cornea* | N/A | 80 | 624 | Non-Cytotoxic |
| RHCIII | 43 μS/cm | 90.0 | 14 | Non-Cytotoxic |
| Prototype | | | | |

*Literature Values

TABLE 10
Summary of Formulations - CollPlant RHCI

| | Collagen | | Two-stage |
|---|---|---|---|
| | Concentration | | Crosslinking |
| Formulation # | (w/w %) | EDC/NHS/NH₂ | (w/v %) |
| 1 | 12% | 0.3/0.3/1 | 0.1% |
| 2* | 12% | 0.3/0.3/1 | 0.1% |
| 3 | 12% | 0.4/0.4/1 | 0.2% |
| 4 | 12% | 0.5/0.5/1 | 0.5% |
| 5 | 12% | 0.4/0.4/1 | 0.2% |
| 6 | 15% | 0.3/0.3/1 | 1% |

*Formulation with collagen powder dissolved in WFI, all the rest formulations with collagen powder dissolved in MES

TABLE 11
Summary of results of key properties of formulations studied -
CollPlant RHCI

| | Light | | |
|---|---|---|---|
| | Transmission | | |
| Formulation # | (400 nm-700 nm) | Refractive Index | T_{d} (° C.) |
| 1 | 95.66% | 1.3657 | 53.80 |
| 2 | 87.58% | 1.3622 | 54.40 |
| 3 | 94.99% | 1.3687 | 57.40 |
| 4 | 94.62% | 1.3643 | 58.90 |
| 5 | 79.92% | 1.3613 | 64.88 |
| 6 | 92.06% | 1.3586 | 76.27 |
| Human Cornea* | >85% | 1.376 | 65.1 |
| RHCIII | 97.50% | 1.3500 | 55.21 |
| Prototype | | | |

*Literature Values

TABLE 12
Summary of results of key properties of formulations studied -
CollPlant RHCI

| | Tensile Strength | Elongation at | Elastic Modulus |
|---|---|---|---|
| Formulation # | (MPa) | break (%) | (MPa) |
| 1 | 0.52 | 22.48 | 4.70 |
| 2 | 0.44 | 21.75 | 3.69 |
| 3 | 0.93 | 20.89 | 8.09 |
| 4 | 0.65 | 15.28 | 6.69 |
| 5 | 0.58 | 15.25 | 6.57 |
| 6 | 0.83 | 12.87 | 9.32 |
| Human Cornea* | 3.8 | 60 | 3-13 |
| RHCIII | 0.35 | 26.62 | 2.82 |
| Prototype | | | |

*Literature Values

TABLE 13
Summary of results of key properties of formulations studied -
CollPlant RHCI

| | Energy at | | |
|---|---|---|---|
| | Break | | |
| | (Toughness, | Suture | Glucose Perm |
| Formulation # | KPa) | Retention (g) | (cm²/s) |
| 1 | 28 | 10.17 | 2.59 × 10⁻⁶ |
| 2 | 23 | 11.01 | 2.29 × 10⁻⁶ |
| 3 | 73 | 14.42 | 2.2 × 10⁻⁶ |
| 4 | 23 | 13.53 | ND |
| 5 | 38 | 18.8 | ND |
| 6 | 36 | ND | 2.77 × 10⁻⁶ |
| Human Cornea* | N/A | >75.6 | 2.5 × 10⁻⁶ |
| RHCIII | ND | 6.65 | 2.96 × 10⁻⁶ |
| Prototype | | | |

*Literature Values
ND: Not Determined

TABLE 14
Summary of results of key properties of formulations studied - CollPlant
RHCI

| | Diafiltration | Water | Collagenase | |
|---|---|---|---|---|
| | Conductivity | Content | Resistance | |
| Formulation # | Value | (%) | (hours) | Cytotoxicity |
| 1 | Collagen Powder | ND | 9 | ND |
| | in MES | | | |
| 2 | Collagen Powder | ND | 13 | ND |
| | in WFI | | | |
| 3 | Collagen Powder | ND | 7.5 | ND |
| | in MES | | | |
| 4 | Collagen Powder | ND | 10 | ND |
| | in MES | | | |
| 5 | 52.7 μS/cm | ND | 10.5 | ND |
| 6 | 165 μS/cm | ND | >144 | Non- |
| | | | | Cytotoxic |
| Human | N/A | 89.16 | 624 | Non- |
| Cornea* | | | | Cytotoxic |
| RHCIII | 43 μS/cm | 84.63 | 14 | Non- |
| Prototype | | | | Cytotoxic |

*Literature Values
ND: Not Determined

### ACRONYMS/DEFINITIONS

T_{d}—denaturing temperature

We claim:

1. A method of making a transparent, double-crosslinked collagen material comprising the steps of:
(a) diafiltering a collagen solution;
(b) lyophilizing the diafiltered collagen solution to produce a collagen powder;
(c) mixing in water or a buffered aqueous solution the collagen powder to obtain a solution with a 3.0% to 23.5%, preferably 12.0% to 15.0%, (w/w) concentration of collagen;
(d) homogenizing and optionally removing air bubbles from the homogenized collagen solution;
(e) mixing the homogenized collagen solution with a 0.002% to 0.01%, preferably 0.006% to 0.008%, (w/v) concentration of crosslinker to form a reaction mixture;
(f) injecting the reaction mixture into a mold and allowing the collagen to crosslink in the mold for up to 24 hours, preferably from 8 to 12 hours, to form a hydrogel;
(g) releasing the hydrogel from the mold and preferably quenching the crosslinking reaction and rinsing the hydrogel;
(h) exposing for up to 24 hours, preferably from 6 to 12 hours, the hydrogel to a buffered aqueous solution containing between 0.1% to 10%, preferably 0.2% to 1.0%, (w/v) concentration of crosslinker, wherein the collagen in the hydrogel blank further crosslinks to form a double-crosslinked collagen material; and
(i) stopping the exposure of the collagen material to the aqueous solution, quenching the crosslinking reaction, and rinsing collagen material for immediate use or for storage.

2. A method of making a transparent, double-crosslinked collagen material comprising the steps of:
(a) diafiltering a collagen solution containing collagen selected from the group consisting of type I, type II, type III, type IV, type V, or type XI collagen, wherein such collagen is non-fibrillar, and is wild type or recombinant;
(b) lyophilizing the diafiltered collagen solution to produce a collagen powder;
(c) mixing in water or a buffered aqueous solution the collagen powder to obtain a solution with a 3.0% to 23.5%, preferably 12.0% to 15.0%, (w/w) concentration of collagen;
(d) homogenizing and optionally centrifuging the collagen solution;
(e) mixing the homogenized collagen solution with a 0.002% to 0.01%, preferably 0.006% to 0.008%, (w/v) concentration of crosslinker to form a reaction mixture;
(f) injecting the reaction mixture into a mold and allowing the collagen to crosslink in the mold for up to 24 hours, preferably from 8 to 12 hours, to form a hydrogel;
(g) releasing the hydrogel from the mold and preferably quenching the crosslinking reaction and rinsing the hydrogel;
(h) placing for up to 24 hours, preferably from 6 to 12 hours, the hydrogel in a buffered aqueous bath containing between 0.1% to 10%, preferably 0.2% to 1.0%, (w/v) concentration of crosslinker, wherein the collagen in the hydrogel blank further crosslinks to form a double-crosslinked collagen material; and
(i) removing the collagen material from the bath, quenching the crosslinking reaction, and rinsing the collagen material for immediate use or for storage.

3. A method of making a transparent, double-crosslinked collagen keratoprosthetic lenticle comprising the steps of:
(a) diafiltering a collagen solution containing collagen selected from the group consisting of type I, type II, type III, type IV, type V, or type XI collagen, wherein such collagen is non-fibrillar, and is wild type or recombinant;
(b) lyophilizing the diafiltered collagen solution to produce a collagen powder;
(c) mixing in water or a buffered aqueous solution the collagen powder to obtain a solution with a 3.0% to 23.5%, preferably 12.0% to 15.0%, (w/w) concentration of collagen;
(d) homogenizing and optionally centrifuging the collagen solution;
(e) mixing the homogenized collagen solution with a 0.002% to 0.01%, preferably 0.006% to 0.008%, (w/v) concentration of crosslinker to form a reaction mixture;
(f) injecting the reaction mixture into a lenticular mold and allowing the collagen to crosslink in the mold for up to 24 hours, preferably from 8 to 12 hours, to form a hydrogel;
(g) releasing the hydrogel from the mold and preferably quenching the crosslinking reaction and rinsing the hydrogel;
(h) placing for up to 24 hours, preferably from 6 to 12 hours, the hydrogel in a buffered aqueous bath containing between 0.1% to 10%, preferably 0.2% to 1.0%, (w/v) concentration of crosslinker, wherein the collagen in the hydrogel blank further crosslinks to form a double-crosslinked keratoprosthetic lenticle; and
(i) removing the keratoprosthetic lenticle from the bath, quenching the crosslinking reaction, and rinsing the keratoprosthetic lenticle for immediate implantation in an eye or for storage.

4. A method according to claim 1, 2, or 3, wherein the crosslinker is EDC/NHS.

5. A method according to claim 1, 2, or 3, wherein the collagen solution used as starting material in subparagraph (a) is prepared by mixing water for injection and acid with collagen powder to prepare a collagen solution with collagen concentration between 0.25-0.35% (w/v), and viscosity between 3000-6000 centiPoise (“cP”), preferably 4500-5500 cP.

6. A method according to claim 1, 2, or 3, wherein the diafiltered collagen solution produced in subparagraph (a) has a conductivity of 43-165 μS/cm, a pH of 3.7-4.5, and a viscosity of 300-900 cP, preferably 650-850 cP.

7. A method according to claim 1, 2 or 3, wherein the pH is controlled in the range of 3.7 to 5.5, preferably in the range of pH 3.7 to 4.5, in subparagraphs (b) to (h).

8. A method according to claim 1, 2, or 3, wherein the pH in subparagraph (e) is controlled in the pH range of 4.0 to 4.5 when the collagen is recombinant human collagen type III, in the pH range of 3.7 to 4.0 when the collagen is recombinant human collagen type I, and in the pH range of 4.0 to 4.5 when the collagen is wild type human collagen type I.

9. A method according to claim 1, 2, or 3, wherein the buffered aqueous solution or buffered aqueous bath is 0.5 M 2-(N-morpholino)ethanesulfonic acid.

10. A method according to claim 1, 2, or 3, wherein the temperature is controlled within a range of 4° C. to 26° C., except in subparagraph (e) the temperature is controlled within a range of 0° C. to 26° C.

11. A method according to claim 1, 2, or 3, wherein the range of temperature during processing is selected from the group consisting of: during diafiltration, within a range of 10° C. to 22°, preferably within a range of 12° C. to 18° C.; during homogenization, within a range of 4° C. to 20°, preferably within a range of 4° C. to 10° C.; during centrifugation, within a range of 4° C. to 20°, preferably within a range of 4° C. to 6° C.; during first stage crosslinking, within a range of 0° C. to 10°, preferably within a range of 4° C. to 6° C.; during second stage crosslinking, within a range of 20° C. to 25°, preferably within a range of 23° C. to 25° C.; and during rinsing, within a range of 20° C. to 25°, preferably within a range of 23° C. to 25° C.

12. A convex or concave lenticular keratoprosthesis produced using the method of claims 1, 2 or 3.

13. A convex or concave lenticular keratoprosthesis produced using the method of claim 1, 2 or 3, wherein the lenticular keratoprosthesis is a corneal onlay, inlay, underlay, or full-thickness cornea.

14. A convex or concave lenticular keratoprosthesis produced using the method of claim 1, 2 or 3, wherein the lenticular keratoprosthesis is implantable.

15. A device produced using the method of claim 1, 2, or 3, and selected from the group consisting of ophthalmic device, tissue scaffold, drug delivery device, and wound dressing.
